SEQUENCE LISTING (3 sequences)

SEQ ID NO: 1 - protein, 557 aa, Homo sapiens
  Protein (1)..(557): Native human soluble thrombomodulin
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 2 - protein, 487 aa, Homo sapiens
  Protein (1)..(487): Solulin (human soluble thrombomodulin analogue)
EPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQLFCNQTACPADCDPNTQASCECPEGYILDDGFICTDIDECENGGFCSGVCHNLPGTFECICGPDSALAGQIGTDCDSGKVDGGDSGAGEPPPSPTPGSTLTPP

SEQ ID NO: 3 - protein, 462 aa, Homo sapiens
  Protein (1)..(462): Human soluble thrombomodulin analogue (SEQ ID NO1 of WO93/25675)
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDC

(19) United States
(12) Patent Application Publication
(10) Pub. No.: US 2012/0270789 A1
(43) Pub. Date: Oct. 25, 2012

(54) TREATMENT OF COAGULOPATHY WITH HYPERFIBRINOLYSIS

(75) Inventors: Karl-Uwe Petersen, Aachen (DE); Michael Ernest Nesheim, Kingston (CA); Daniel Nesheim, Kingston (CA); Jonathan Herbert Foley, Kingston (CA)

(73) Assignee: PAION Deutschland GmbH, Aachen (DE)

(21) Appl. No.: 13/377,497

(22) PCT Filed: Jun. 12, 2009
(86) PCT No.: PCT/EP2009/004218

§ 371 Date: Jul. 11, 2012

(51) Int. Cl. A61K 38/17; A61P 7/04; C07K 14/705

(52) U.S. Cl. 514/14.7; 530/350

(57) ABSTRACT

The present invention relates to the use of thrombomodulin analogues for the manufacture of a medicament for the treatment of coagulopathy with hyperfibrinolysis, such as haemophilia disorders. These thrombomodulin analogs exhibit at therapeutically effective dosages an antifibrinolytic effect. Novel protein modifications together with methods for their identification are disclosed.

The invention relates to the field of coagulopathy with hyperfibrinolysis. More particularly, this invention relates to the treatment of haemophila diseases such as haemophilia A or haemophilia B.

Haemophilia is a group of hereditary genetic disorders that impair the body's ability to control blood clotting or coagulation, which is used to stop bleeding when a blood vessel is broken. Haemophilia A, the most common form, results from a mutation in the gene for Factor VIII; haemophilia B, also known as Christmas disease, results from a mutation in the gene for Factor IX. Haemophilia B, like haemophilia A, is X-linked and accounts for approximately 12% of haemophilia cases. The symptoms are identical to those of haemophilia A: excessive bleeding upon injury; and spontaneous bleeding, especially into weight-bearing joints, soft tissues, and mucous membranes. Repeated bleeding into joints results in haemarthrosis, causing painful crippling arthropathy that often necessitates joint replacement. Haematomas in soft tissues can result in pseudo tumors composed of necrotic coagulated blood; they can obstruct, compress, or rupture into adjacent organs and can lead to infection. Once formed the haematomas are difficult to treat, even with surgery. Recovery of nerves after compression is poor, resulting in palsy. Those bleeding episodes that involve the gastrointestinal tract, central nervous system, or airway/retroperitoneal space can lead to death if not detected. Intracranial bleeding is a major cause of death in haemophiliacs.

There are estimated to be 100,000 cases of congenital haemophilia in the United States. Of these, approximately 20,000 are cases of haemophilia B, the blood of such patients being either totally devoid of Factor IX or seriously deficient in plasma Factor IX component. The disease therefore exists in varying degrees of severity, requiring therapy anywhere from every week up to once or twice a year. The completely deficient cases require replacement therapy once every week; the partially deficient cases require therapy only when bleeding episodes occur, which may be as seldom as once a year. The bleeding episodes in congenital, partially deficient cases are generally caused by a temporarily acquired susceptibility rather than by injury alone. Intravenous injection of a sufficiently large amount of fresh plasma, or an equivalent amount of fresh blood temporarily corrects the defect of a deficient subject. The beneficial effect often lasts for two or three weeks, although the coagulation defect as measured by in vitro tests on the patient's blood appears improved for only two or three days.

Such therapy with fresh plasma or fresh blood is effective but it has several serious drawbacks: (1) it requires ready availability of a large amount of fresh plasma; (2) requires hospitalization for the administration of the plasma; (3) a great many of the patients become sensitized to repeated blood or plasma infusions and ultimately encounter fatal transfusion reactions; (4) at best plasma can only partially alleviate the deficiency; and (5) prolonged treatment or surgery is not possible because the large amounts of blood or plasma which are required will cause acute and fatal edema.

An improved therapy includes intravenous replacement therapy with Factor VIII or Factor IX concentrates. However, also this therapy suffers from several disadvantages: (1) when treating major bleeding episodes tissue damage remains even after prompt detection and treatment; (2) a great many of the patients become refractory to the coagulation factors and develop inhibitory antibodies against the coagulation factors (so called haemophilia with inhibitors); (3) despite the improved virus inactivation methods there is still an increased risk of contamination with fatal viruses such as HIV and hepatitis C (it is estimated that more than 50% of the haemophilia population, over 10,000 people, contracted HIV from the tainted blood supply in the USA); (4), the isolated and especially the recombinant clotting factors are very expensive and not generally available in the developing world.

A treatment or prevention of bleeding beyond a replacement therapy is a challenge because bleeding in haemophilia is a complex pathophysiological process that may be attributable to triple defects: (1) a reduced thrombin generation via the extrinsic pathway at low tissue factor concentration, (2) a reduced secondary burst of thrombin generation via the intrinsic pathway, and (3) a defective downregulation of the fibrinolytic system by the intrinsic pathway.

The fact that a reduced thrombin generation results in a reduced clotting propensity and therefore an increased risk of bleeding is generally accepted. However, work in the past decade indicates that also a defective downregulation of the fibrinolysis may play a role in haemophilia. As a result haemophila can be also classified as a coagulopathy with hyperfibrinolysis.

A recent publication supports this assumption by showing in vitro that when a clot is formed in Factor VIII depleted plasma (FVIII-DP) and supplemented with tissue plasminogen activator tPA, fibrinolysis is not adequately downregulated and as a result the clot lyses prematurely (Braze and Higuchi, Blood 1996, 88; 3815-3823; Mosnier et al.; Thromb. Haemost. 2001, 86: 1035-1039). Furthermore, it could be shown that this “premature lysis” is due to reduced or absent activation of thrombin-activatable fibrinolysis inhibitor (TAFI) (Broze and Higuchi, 1996) and that in FVIII-DP, an activated TAFI containing mixture increases clot lysis time. It was concluded that stabilized TAFI can be used for the treatment of haemophilia (WO02/099098).

TAFI plays a crucial role in the downregulation of fibrinolysis, which is required for formation of stable clots. TAFI also known as plasma procarboxypeptidase B2 or procarboxypeptidase U is a plasma zymogen that, when exposed to the thrombin-thrombomodulin complex, is converted by proteolysis at Arg⁹² to a basic carboxypeptidase (TAFIa or activated TAFI) that inhibits fibrinolysis. It potently attenuates fibrinolysis by removing the C-terminal lysine and arginine residues from fibrin which are important for the binding and activation of plasminogen.

As discussed above thrombomodulin (TM) in complex with thrombin is responsible for the TAFI activation. Thrombomodulin is a membrane protein that acts as a thrombin receptor on the endothelial cells lining the blood vessels. Thrombin is a central enzyme in the coagulation cascade, which converts fibrinogen to fibrin, the matrix clots are made of. Initially, a local injury leads to the generation of small amounts of thrombin from its inactive precursor prothrombin. Thrombin, in turn, activates platelets and, second, certain coagulation factors including factors V and VIII. The latter action gives rise to the so-called thrombin burst, a massive activation of additional prothrombin molecules, which finally results in the formation of a stable clot.

When bound to thrombomodulin, however, the activity of thrombin is changed in direction: A major feature of the thrombin-thrombomodulin complex is its ability to activate protein C, which then downregulates the coagulation cascade by proteolytically inactivating the essential cofactors Factor Va and Factor VIIIa (Esmon et al., Ann. N.Y. Acad. Sci. (1991), 614:30-43), thus affording anticoagulant activity. The thrombin-thrombomodulin complex is also able to activate the thrombin-activatable fibrinolysis inhibitor (TAFI), which then antagonizes fibrinolysis (see above).

Mature human TM is composed of a single polypeptide chain of 559 residues and consists of five domains: an aminoterminal “lectin-like” domain, an “6 EGF-like repeat domain” comprising six epidermal growth factor (EGF)-like repeats, an O-glycosylation domain, the transmembrane domain and a cytoplasmic domain with following localisation (amino acid position as given in SEQ ID NO:1 or SEQ ID NO:3):

| Approx. amino acid position | Domain |
|---|---|
| −18-−1 | Signal sequence |
| 1-226 | N-terminal domain (lectin-like) |
| 227-462 | 6 EGF-like repeat domains |
| 463-497 | O-linked Glycosylation |
| 498-521 | Transmenbrane domain |
| 522-557 | Cytoplasmic domain |

Various structure-function studies using proteolytic fragments of rabbit TM or deletion mutants of recombinant human TM have localized its activity to the last three EGF-like repeats. The smallest mutant capable of efficiently promoting TAFI activation contained residues including the c-loop of epidermal growth factor-3 (EGF3) through EGF6. This mutant is 13 residues longer than the smallest mutant that activates C; the latter consisted of residues from the interdomain loop connecting EGF3 and EGF4 through EGF6.

As discussed above the replacement therapy for treating coagulation disorders such as haemophilia does not meet the medical needs. Importantly, no drug besides the coagulation factors used for the replacement therapy is available which can prevent or treat haemophilia patients.

Thus, despite the long-standing need for the development of therapies to prevent or treat coagulopathy with hyperfibrinolysis, in particular haemophilia, progress has been slow, and therapeutics that are safe and effective are still missing.

Thus, it is the objective of the present invention to provide novel means for the treatment of coagulopathy with hyperfibrinolysis.

This objective is solved by providing a medicament for the treatment of coagulopathy with hyperfibrinolysis in a mammal, in particular in humans, comprising a thrombomodulin analogue exhibiting at therapeutically effective dosages an antifibrinolytic effect.

This novel approach is based on the surprising findings that a thrombomodulin can be modified in a way that it exhibits an antifibrinolytic activity that prevail its profibrinolytic activity even at high plasma concentrations, in particular at concentrations of more than 15 nM, in particular more than 20, 30, 40 or 50 nM (at least up to 100 nM). Hence these TM analogues exhibit an antifibrinolytic effect, and are thus suitable for the use according to the invention.

This antifibrinolytic effect was shown in plasma from haemophilia patients (which is depleted for Factor VIII; FVIII-DP). Therewith it was demonstrated that such a thrombomodulin analogue can be used as a therapeutic.

So far the therapeutic use of thrombomodulin for the treatment of haemophilia was not regarded as a real option because it was known from rabbit lung thrombomodulin (rITM) that it always has both anti- and profibrinolytic activities even at rather low concentrations (see Mosnier and Bouma; Arterioscler. Thromb. Vasc. Biol. 2006; 26: 2445-2453; especially FIG. 5). At plasma concentrations of less than 15 nM rITM increased clot lysis time whereas at plasma concentrations greater than 15 nM a marked decrease in lysis time was demonstrated (Mosnier et al., 2001, Mosnier and Bouma, 2006) with a profibrinolytic effect as the final result. This profibrinolytic effect at higher concentrations prohibits any therapeutical use in haemophilia since a potential overdosing or individual variabilites in susceptibility would fatally aggravate, prolong or even cause bleeding events.

According to the invention various options exist which lead to TM analogues that exhibit an antifibrinolytic effect and thus are suitable for the treatment according to the invention.

In one embodiment thrombomodulin analogues can be used with reduced binding affinity to thrombin. Consequently they can prolong the clot lysis in normal plasma and FVIII-DP, e.g. up to 100 nM (FIG. 4).

The importance of these findings is that these thrombomodulin analogues exhibit an antifibrinolytic effect without a deleterious profibrinolytic effect even at high concentrations. This concentration exceeds by far the therapeutically effective dosages. Therefore the TM analogues enable the treatment of coagulopathy with hyperfibrinolysis.

Without bound to this theory the inventors have shown that this therapeutic potential of the TM analogues can be explained by the fact that they show a markedly reduced affinity towards thrombin. This was shown by Bajzar et al. (J. Biol. Chem 1996; 271: 16603-16608) who found a K_{D} value of 23 nM in contrast to the K_{D} value of 0.2 nM observed for the binding between thrombin and rabbit lung thrombomodulin (Esmon et al., Ann. NY. Acad. Sci. 1986, 485: 215-220).

Hence, according to one embodiment of the invention thrombomodulin analogues can be used for the treatment of coagulopathy with hyperfibrinolysis which have a reduced binding affinity towards thrombin compared to the rabbit lung thrombomodulin.

In particular, a thrombomodulin analogue can be used which exhibits a K_{D} for thrombin binding of more than 0.2 nM, preferably more than 1 nM, 2 nM, 4 nM, 5 nM, 7.5 nM, 10 nM, 12.5 nM, 15 nM, 17.5 nM, 20 nM, 22.5 nM, or 25 nM, and more preferably a K_{D} value in a range between 10 and 30 nM or more.

In a further embodiment of the invention, the reduced profibrinolytic activity of a thrombomodulin analogue can be due to a reduced ability to activate protein C (so called “cofactor activity”). Since the protein C activation results in an upregulation of fibrinolysis (Mosnier et al., 2001) a reduced cofactor activity will prolong the clot lysis time. The person skilled in art knows several strategies to reduce the cofactor activity of thrombomodulin, such as e.g. changes in the glycosylation, secondary or tertiary structure of the protein or preferably changes in the primary structure e.g. by mutation of one or more amino acids.

In a yet another embodiment TM analogues can be used which have a reduced cofactor activity compared to the thrombomodulin analogue TM_{E}M388L, where TM_{E} denotes to an analogue consisting of the six EGF domains only.

According to the invention a thrombomodulin analogue can also be used which has an increased ability to activate TAFI (so called “TAFI activation activity”) since TAFI activation results in a downregulation of fibrinolysis (Mosnier and Bouma, 2006). For the person skilled in art there are several strategies to increase the TAFI activation activity by thrombomodulin such as changes in the glycosylation, secondary or tertiary structure of the protein or preferably changes in the primary structure e.g. by mutation of one or more amino acids.

Particularly, this invention also provides for a thrombomodulin analogue which has a significantly increased ratio of TAFI activation activity to cofactor activity compared to the thrombomodulin analogue TM_{E}M388L.

Notably, according to the invention the TM analogue used for the treatment of coagulopathy has one or more of the above described features, namely:
a) a binding affinity towards thrombin that is decreased compared to the rabbit lung thrombomodulin, and/or a binding affinity towards thrombin with a k_{D} value of more than 0.2 nM;
b) a reduced cofactor activity compared to cofactor activity of the TM analogue TMEM388L, or
c) an increased ratio of TAFI activation activity to cofactor activity as compared to the TM analogue TM_{E}M388L.

In an embodiment of the invention, thrombomodulin can be used to treat human patients with any coagulopathy that occurs with a prominently or even slightly reduced fibrinolysis compared to normal subjects. In particular the following diseases can be treated with the thrombomodulin analogue: haemophilia A, haemophilia B, haemophilia C, von Willebrandt disease (vWD), acquired von Willebrandt disease, Factor X deficiency, parahaemophilia, hereditary disorders of the clotting factors I, II, V, or VII, haemorrhagic disorder due to circulating anticoagulants (including autoantibodies against coagulation factors such as Factor VIII) or acquired coagulation deficiency.

It will be understood that the therapeutic success that can be maintained or achieved by the treatment of the invention depends on the nature and the degree of the disease in any particular patient.

Specific embodiments of the invention relate to the prophylactic treatment of coagulopathy to prevent bleeding or to the acute treatment when bleeding occurs (“on demand”). The bleeding events to be treated with the thrombomodulin analogue can occur in every organ or tissue in the organism, most importantly in the central nervous system e.g. as intracranial bleeding, in the joints, the muscles, the gastrointestinal tract, the respiratory tract, the retroperitoneal space or soft tissues.

For the preventive treatment the TM analogue can be given to the patient at regular intervals over an extended period. However, also multiple dosing for a rather restricted time period (“subchronic treatment”) is possible.

In one embodiment of the invention the thrombomodulin analogue is given in advance of a higher bleeding risk, e.g. a surgery or a tooth extraction.

In a further embodiment of the invention the thrombomodulin analogue is administered to patients that are refractory to standard therapy such as the transfusion of blood or plasma or the replacement therapy using coagulation factors.

According to the invention the TM analogue can be administered in multiple doses preferably once daily but also bidaily, or every third, fourth, fifth, sixth or seven days over a total time period of less than one week to four weeks, more preferably as chronic administration. Thus, according to the invention a pharmaceutical composition is provided, which is suitable for allowing a multiple administration of the thrombomodulin analogue.

The TM analogue is given preferably non-orally as a parenteral application e.g. by intravenous or subcutaneous application. An intravenous or subcutaneous bolus application is possible. Thus, according to the invention a pharmaceutical composition is provided, which is suitable for a parenteral administration of thrombomodulin.

In one embodiment of the invention the thrombomodulin analogue is a soluble TM analogue, in particular a TM analogue where the cytoplasmic domain is deleted and the transmembrane domain is completely or partially deleted.

In a preferred embodiment of the invention the thrombomodulin analogue comprises at least one structural domain selected from the group containing EGF3, EGF4, EGF5, or EGF6, preferably the EGF domains EGF1 to EGF6, more preferably the EGF domains EGF3 to EGF6 and most preferably the EGF domains EGF4 to EGF6 and particularly the fragment including the c-loop of epidermal growth factor-3 (EGF3) through EGF6.

Various forms of soluble thrombomodulin are known to the skilled person, e.g. the so called ART-123 developed by Asahi Corporation (Tokyo, Japan) or the recombinant soluble human thrombomodulin Solulin, currently under development by PAION Deutschland GmbH, Aachen (Germany). The recombinant soluble thrombomodulin, i.e. a soluble thrombomodulin without a modification of the amino acid sequence, is subject of the Asahi patent EP0 312 598.

Solulin is a soluble, as well as protease and oxidation-resistant analogue of human thrombomodulin and thus exhibits a long life in viva Solulin's main feature lies in its broad mechanism of action since it not exclusively inhibits thrombin. It also activates TAFI and the natural protein C/protein S pathway. As a result of its reduced thrombin binding Solulin inhibits fibrinolysis even up to high concentrations.

Solulin is inter alia subject of the European patent 0 641 215 B1, EP 0 544 826 B1 as well as EP 0 527 821 B1. Solulin contains modifications compared to the sequence of native human thrombomodulin (SEQ. ID NO. 1) at the following positions: G −3V, Removal of amino acids 1-3, M388L, R456G, H457Q, S474A and termination at P490. This numbering system is in accordance with the native thrombomodulin of SEQ. ID NO. 1 and SEQ ID NO:3. The sequence of Solulin as one preferred embodiment of the invention is shown in SEQ ID NO: 2.

However, notably, according to the invention also thrombomodulin analogues can be used, which comprise only one or more of the above mentioned properties, or of the properties outlined in the above mentioned European patent documents EP 0 544 826 B1, EP 0 641 215 B1 and EP 0 527 821 B1.

Particularly preferred thrombomodulin analogues applicable according to the invention are those that have one or more of the following characteristics:
a) they exhibit oxidation resistance,
b) they exhibit protease resistance,
c) they have homogeneous N- or C-termini,
d) they have been post-translationally modified, e.g., by glycosylation of at least some of the glycosylation sites of native thrombomodulin (SEQ ID NO: 1),
e) they have linear double-reciprocal thrombin binding properties,
f) they are soluble in aqueous solution in relatively low amounts of detergents and typically lack a transmembrane sequence,
g) they are lacking a glycosaminoglycan chain.

The manufacture of these analogues used in this invention is disclosed in the above mentioned European patent documents.

In one embodiment of the invention only the six EGF domains of Solulin can be used, in particular a Solulin fragment consisting of the EGF4 to EGF6 domain.

In an embodiment a thrombomodulin analogue with reduced cofactor activity as known from the WO93/25675 can be used. A series of thrombomodulin analogues is described herein having about 50% or less of the cofactor activity of the control human soluble thrombomodulin (TM_{E}M388L).

More particularly said thrombomodulin analogues upon binding to thrombin, exhibit a modified cofactor activity as compared to binding with TM_{E}M388L of less than or equal to 50%, said analogue having amino acid substitutions at one or more positions corresponding to the amino acid position as given in SEQ ID NO:1 or SEQ ID NO:3:
aa) ³⁴⁹Asp;
ab) ³⁵⁵Asn;
ac) ³⁵⁷Glu;
ad) ³⁵⁸Tyr;
ae) ³⁵⁹Gln;
af) ³⁶³Leu;
ai) ³⁶⁸Tyr;
aj) ³⁷¹Val;
ak) ³⁷⁴Glu;
al) ³⁷⁶Phe;
am) ³⁸⁴His;
an) ³⁸⁵Arg;
ba) ³⁸⁷Gln;
bb) ³⁸⁹Phe;
bc) ³⁹⁸Asp;
bd) ⁴⁰⁰Asp;
be) ⁴⁰²Asn;
bf) ⁴⁰³Thr;
bg) ⁴⁰⁸Glu;
bh) ⁴¹¹Glu;
bi) ⁴¹³Tyr;
bj) ⁴¹⁴Ile;
bk) ⁴¹⁵Leu;
bl) ⁴¹⁶Asp;
bm) ⁴¹⁷Asp;
bn) ⁴²⁰Ile;
ca) ⁴²³Asp;
cb) ⁴²⁴Ile;
cc) ⁴²⁵Asp;
cd) ⁴²⁶Glu;
ce) ⁴²⁸Glu;
cf) ⁴²⁹Asp;
cg) ⁴³²Phe;
ch) ⁴³⁴Ser;
ci) ⁴³⁶Val;
cj) ⁴³⁸His;
ck) ⁴³⁹Asp;
cl) ⁴⁴⁰Leu;
cm) ⁴⁴³Thr;
cn) ⁴⁴⁴Phe;
co) ⁴⁴⁵Glu;
cp) ⁴⁵⁶Arg;
cq) ⁴⁵⁸Ile; or
cr) ⁴⁶¹Asp

Most preferred are TM analogues with only one of the above listed substitutions. For convenience the designation to the left, e.g. aa) are identical for each modified site. The first letter represents the EGF domain, where a is EGF4; b is EGF5 and c is EGF6. The second letter represents the relative position of the modification with regard to other residues in the listing. Also provided herein are nucleic acids encoding the TM analogues described above.

The following analogues constitute a preferred subset of the above given analogues wherein the analogues have 25% or less of the cofactor activity of the control, TM_{E}M388L. These analogues have one or more amino acid substitutions, preferably only one (amino acid position as given in SEQ ID NO:1 or SEQ ID NO:3):
aa) ³⁴⁹Asp;
ac) ³⁵⁷Glu;
ad) ³⁵⁸Tyr;
ae) ³⁵⁹Gln;
aj) ³⁷¹Val;
ak) ³⁷⁴Glu;
al) ³⁷⁶Phe;
bc) ³⁹⁸Asp;
bd) ⁴⁰⁰Asp;
be) ⁴⁰²Asn;
bg) ⁴⁰⁸Glu;
bi) ⁴¹³Tyr;
bj) ⁴¹⁴Ile;
bk) ⁴¹⁵Leu;
bl) ⁴¹⁶Asp;
bm) ⁴¹⁷Asp;
bo) ⁴²³Asp;
bp) ⁴²⁴Ile;
bq) ⁴²⁵Asp;
cd) ⁴²⁶Glu;
ce) ⁴²⁹Asp;
ck) ⁴³⁹Asp;
cn) ⁴⁴⁴Phe; or
cr) ⁴⁶¹Asp.

The modifications set forth above with regard to protease activity, aliphatic substitutions, oxidation resistance and uniform termini are also applicable for the above analogues having less than 50% of the cofactor activity of the control.

Preferred are those listed above having less than 30% of the activity of the control. These analogues are represented by mutations in domain 4. These analogues have one or more amino acid substitutions, preferably only one (amino acid position as given in SEQ ID NO:1 or SEQ ID NO:3):
aa) ³⁴⁹Asp;
ac) ³⁵⁷Glu;
ad) ³⁵⁸Tyr;
ae) ³⁵⁹Gln;
aj) ³⁷¹Val; or
al) ³⁷⁶Phe.

There are also described herein analogues having an essentially unmodified K_{D} value compared to TMEM388L. EGF5 and EGF6 are known to play an important role in high affinity binding to thrombin, whereas EGF4 with a less critical role in binding is critical for conferring cofactor activity to the TM/thrombin complex. For this reason those analogues having modifications in the EGF repeats 5 and 6 can have almost the same cofactor activity but a reduced K_{D} compared to TM_{E}M388L, e.g. (S406A). Analogues having modifications in the EGF repeats 5 and 6 which resulted in reduced cofactor activity are listed below. These analogues have one or more amino acid substitutions, preferably only one (amino acid position as given in SEQ ID NO:1 or SEQ ID NO:3):
bc) ³⁹⁸Asp;
bd) ⁴⁰⁰Asp;
be) ⁴⁰²Asn;
bf) ⁴⁰³Thr;
bg) ⁴⁰⁸Glu;
bi) ⁴¹³Tyr;
bj) ⁴¹⁴Ile;
bk) ⁴¹⁵Leu;
bl) ⁴¹⁶Asp;
bm) ⁴¹⁷Asp;
ca) ⁴²³Asp;
cb) ⁴²⁴Ile;
cc) ⁴²⁵Asp;
cd) ⁴²⁶Glu;
cf) ⁴²⁹Asp;
ck) ⁴³⁹Asp;
cn) ⁴⁴⁴Phe; or
cr) ⁴⁶¹Asp

The above analogues may also grouped by their respective domains (i.e., EGF4, EGF5 or EFG6) as well as by their respective relative activity. For example the analogues of EGF4 having approximately 50% of the control cofactor activity are (amino acid position as given in SEQ ID NO:1 or SEQ ID NO:3):
ac) ³⁴⁹Asp;
ac) ³⁵⁵Asn;
ac) ³⁵⁷Glu;
ad) ³⁵⁸Tyr;
ae) ³⁵⁹Gln;
af) ³⁶³Leu;
ai) ³⁶⁸Tyr;
aj) ³⁷¹Val;
ak) ³⁷⁴Glu;
al) ³⁷⁶Phe;
am) ³⁸⁴His; or
an) ³⁸⁵Arg.

Most preferred are TM analogues with only one of the above listed substitutions.

Those in EGF4 having less than 25% of the cofactor activity of the control are (amino acid position as given in SEQ ID NO:1 or SEQ ID NO:3):
aa) ³⁴⁹Asp;
ac) ³⁵⁷Glu;
ad) ³⁵⁸Tyr;
ae) ³⁵⁹Gln;
aj) ³⁷¹Val; or
al) ³⁷⁶Phe.

Most preferred are TM analogues with only one of the above listed substitutions.

In EGF5, the following modifications resulted in analogues having at least a 50% reduction in cofactor activity (amino acid position as given in SEQ ID NO:1 or SEQ ID NO:3):
bc) ³⁹⁸Asp;
bd) ⁴⁰⁰Asp;
be) ⁴⁰²Asn;
bf) ⁴⁰³Thr;
bg) ⁴⁰⁸Glu;
bh) ⁴¹¹Glu;
bi) ⁴¹³Tyr;
bj) ⁴¹⁴Ile;
bk) ⁴¹⁵Leu;
bl) ⁴¹⁶Asp;
bm) ⁴¹⁷Asp, or
bn) ⁴²⁰Ile.

Most preferred are TM analogues with only one of the above listed substitutions. Among these analogues are those where the analogues have an essentially unmodified kCat/Km compared to TM_{E}M388L.

In EGF5, the analogues can be further subgrouped according to those modifications resulted in analogues having at least a 75% reduction in cofactor activity (amino acid position as given in SEQ ID NO:1 or SEQ ID NO:3):
bc) ³⁹⁸Asp;
bd) ⁴⁰⁰Asp;
be) ⁴⁰²Asn;
bg) ⁴⁰⁸Glu;
bi) ⁴¹³Tyr;
bj) ⁴¹⁴Ile;
bk) ⁴¹⁵Leu;
bl) ⁴¹⁶Asp; or
bm) ⁴¹⁷Asp.

Most preferred are TM analogues with only one of the above listed substitutions. Among these analogues are those with essentially unmodified kCat/Km compared to TMEM388L. Nucleic acids encoding the above analogues are also provided.

With regard to EGF6 the groups are provided below. Those having a cofactor activity of less than 50% of the control are (amino acid position as given in SEQ ID NO:1 or SEQ ID NO:3):
ca) ⁴²³Asp;
cb) ⁴²⁴Ile;
cc) ⁴²⁵Asp;
cd) ⁴²⁶Glu;
ce) ⁴²⁸Glu;
cf) ⁴²⁹Asp;
cg) ⁴³²Phe;
ch) ⁴³⁴Ser;
ci) ⁴³⁶Val;
cj) ⁴³⁸His;
ck) ⁴³⁹Asp;
cl) ⁴⁴⁰Leu;
cm) ⁴⁴³Thr;
cn) ⁴⁴⁴Phe;
co) ⁴⁴⁵Glu;
cp) ⁴⁵⁶Arg;
cq) ⁴⁵⁸Ile; or
cr) ⁴⁶¹Asp.

Most preferred are TM analogues with only one of the above listed substitutions.

Those having a cofactor activity of less than 25% of the control are (amino acid position as given in SEQ ID NO:1 or SEQ ID NO:3):
ca) ⁴²³Asp;
cb) ⁴²⁴Ile;
aa) ⁴²⁵Asp;
cd) ⁴²⁶Glu;
cf) ⁴²⁹Asp;
ck) ⁴³⁹Asp;
cn) ⁴⁴⁴Phe; or
cr) ⁴⁶¹Asp.

Most preferred are TM analogues with only one of the above listed substitutions. The preferred analogues are those set forth above with additional modifications for solubility, protease resistance, oxidation resistance as well as uniform terminal ends. The nucleic acids encoding these analogues are also a part of the claimed invention. As with the other groups, these analogues include those wherein said analogue has an essentially unmodified kCat/Km compared to TM_{E}M388L.

The analogues can be further subgrouped according to those possessing a modified amino acid at a certain position, wherein said analogue has essentially equivalent K_{D} for thrombin compared to an analogue having at said position the native residue, wherein said position corresponds to (amino acid position as given in SEQ ID NO:1 or SEQ ID NO:3):
ca) ³⁴⁹Asp;
cb) ³⁵⁵Asn;
ac) ³⁵⁷Glu;
ad) ³⁵⁸Tyr; or
ae) ³⁵⁹Gln.

Most preferred are TM analogues with only one of the above listed substitutions. These analogues may have a modified kCat/Km of less than 30% of the control.

The following sites embrace described analogues having a modified K_{D} or kCat/Km compared to an analogue having at said position the native residue, wherein said position corresponds to (amino acid position as given in SEQ ID NO:1 or SEQ ID NO:3):
af) ³⁶³Leu;
aj) ³⁷¹Val;
ak) ³⁷⁴Glu;
al) ³⁷⁶Phe;
am) ³⁸⁴His;
an) ³⁸⁵Arg;
bc) ³⁹⁸Asp;
bd) ⁴⁰⁰Asp; or
be) ⁴⁰²Asn.

Most preferred are TM analogues with only one of the above listed substitutions. These further include those analogues having both a modified K_{D} and kCat/Km, especially those having been modified by at least 20%.

The following sites describe analogues having a lower cofactor activity and a K_{D} or kCat/Km that is essentially equivalent when compared to an analogue having at said position the native residue, wherein said position corresponds to (amino acid position as given in SEQ ID NO:1 or SEQ ID NO:3):
bg) ⁴⁰⁸Glu;
bh) ⁴¹¹Glu;
bi) ⁴¹³Tyr;
bj) ⁴¹⁴Ile;
bk) ⁴¹⁵Leu;
bl) ⁴¹⁶Asp;
bm) ⁴¹⁷Asp;
bn) ⁴²⁰Ile;
ca) ⁴²³Asp;
cb) ⁴²⁴Ile;
cc) ⁴²⁵Asp;
cd) ⁴²⁶Glu;
ce) ⁴²⁸Glu;
cf) ⁴²⁹Asp;
cg) ⁴³²Phe;
ch) ⁴³⁴Ser;
ci) ⁴³⁶Val;
cj) ⁴³⁸His;
ck) ⁴³⁹Asp;
cl) ⁴⁴⁰Leu;
cm) ⁴⁴³Thr;
cn) ⁴⁴⁴Phe;
co) ⁴⁴⁵Glu;
cp) ⁴⁵⁶Arg;
cq) ⁴⁵⁸Ile; or
cr) ⁴⁶¹Asp.

Most preferred are TM analogues with only one of the above listed substitutions.

The following positions describe a subgrouping of those modifications which resulted in at least a 75% reduction in cofactor activity yet essentially little change in kcat/Km (amino acid position as given in SEQ ID NO:1 or SEQ ID NO:3):
bg) ⁴⁰⁸Glu;
bi) ⁴¹³Tyr;
bj) ⁴¹⁴Ile;
bk) ⁴¹⁵Leu;
bl) ⁴¹⁶Asp;
bm) ⁴¹⁷Asp;
ca) ⁴²³Asp;
cb) ⁴²⁴Ile;
cc) ⁴²⁵Asp;
cd) ⁴²⁶Glu;
cf) ⁴²⁹Asp;
ck) ⁴³⁹Asp;
cn) ⁴⁴⁴Phe; or
cr) ⁴⁶¹Asp.

Most preferred are TM analogues with only one of the above listed substitutions. A further subgrouping can be made of the above modifications wherein the K_{D} for thrombin is modified by at least 30%.

This invention further provides for methods. More specifically there is described herein a method useful for screening for analogues of thrombomodulin which exhibit a modified Kd for thrombin binding, comprising the steps of:
a) making an amino acid substitution at a position (amino acid position as given in SEQ ID NO:1 or SEQ ID NO:3):
   bg) ⁴⁰⁸Glu;
   bi) ⁴¹³Tyr;
   bj) ⁴¹⁴Ile;
   bk) ⁴¹⁵Leu;
   bl) ⁴¹⁶Asp;
   bm) ⁴¹⁷Asp;
   bn) ⁴²⁰Ile;
   ca) ⁴²³Asp;
   cb) ⁴²⁴Ile;
   cc) ⁴²⁵Asp;
   cd) ⁴²⁶Glu;
   ce) ⁴²⁸Glu;
   cf) ⁴²⁹Asp;
   cg) ⁴³²Phe;
   ch) ⁴³⁴Ser;
   ci) ⁴³⁶Val;
   cj) ⁴³⁸His;
   ck) ⁴³⁹Asp;
   cl) ⁴⁴⁰Leu;
   cm) ⁴⁴³Thr;
   cn) ⁴⁴⁴Phe;
   co) ⁴⁴⁵Glu;
   cp) ⁴⁵⁶Arg;
   cq) ⁴⁵⁸Ile;
   cr) ⁴⁶¹Asp; and
b) comparing the K_{D} for thrombin to a control molecule.

As used within these methods TM analogues with only one amino acid substitutions are preferred. Various embodiments of this invention include those wherein said K_{D} is modified by at least 33%, or where said modification is an amino acid substitution, or wherein said control molecule is TM_{E}M388L. A preferred grouping of modifications for use in the method are (amino acid position as given in SEQ ID NO:1 or SEQ ID NO:3):
bg) ⁴⁰⁸Glu;
bi) ⁴¹³Tyr;
bj) ⁴¹⁴Ile;
bk) ⁴¹⁵Leu;
bl) ⁴¹⁶Asp;
bm) ⁴¹⁷Asp;
ca) ⁴²³Asp;
cb) ⁴²⁴Ile;
cc) ⁴²⁵Asp;
cd) ⁴²⁶Glu;
cf) ⁴²⁹Asp;
ck) ⁴³⁹Asp;
cn) ⁴⁴⁴Phe; or
cr) ⁴⁶¹Asp.

As used within these methods TM analogues with only one amino acid substitutions are preferred.

An another method is described herein which is useful for screening for analogues of thrombomodulin which possess a modified cofactor activity upon binding to thrombin, comprising the steps of:
a) making an amino acid substitution at a position (amino acid position as given in SEQ ID NO:1 or SEQ ID NO:3):
   aa) ³⁴⁹Asp;
   bb) ³⁵⁵Asn;
   ac) ³⁵⁷Glu;
   ad) ³⁵⁸Tyr;
   ae) ³⁵⁹Gln; and
b) comparing the rate of cofactor activity upon binding to thrombin with the rate of a control molecule.

As used within these methods TM analogues with only one amino acid substitutions are preferred.

In a preferred embodiment of the invention the thrombomodulin analogue has a modification of the phenylalanine residue at position 376 (SEQ ID NO:1 or SEQ ID NO:3). This residue can be chemically or biochemically modified or deleted by methods that are well known for the person skilled in art. The phenylalanine residue is preferably substituted with an aliphatic amino acid, more preferably with glycine, alanine, valine, leucine, or isoleucine and most preferably substituted with alanine. It was demonstrated that a substitution of Phe³⁷⁶ by alanine (“F376A”) substantially decreased the cofactor activity of the thrombomodulin analogue while preserving the TAFI activation activity (see FIG. 7). As a result the F376A-TM analogue has an increased ratio of TAFI activation activity versus cofactor activity.

In a further embodiment of the invention the thrombomodulin analogue has a modification of the glutamine residue at position 387 (SEQ ID NO:1 or SEQ ID NO:3). The glutamine residue is preferably substituted with the following amino acids, ordered in decreasing cofactor activity of the resulting mutant Gln387X-TM analogue (see FIG. 8A): Met, Thr, Ala, Glu, His, Arg, Ser, Val, Lys, Gly, Ile, Tr, Tyr, Leu, Asn, Phe, Asp, Cys.

In another embodiment of the invention the thrombomodulin analogue has a modification of the methionine residue at position 388 (SEQ ID NO:1 or SEQ ID NO:3). The methionine residue is preferably substituted with the following amino acids, ordered in decreasing cofactor activity of the resulting mutant Met388X-TM analogue (see FIG. 8B): Gln, Tyr, Ile, Phe, His, Arg, Pro, Val, Thr, Ser, Ala, Trp, Asn, Lys, Gly, Glu, Asp, Cys.

In a further embodiment of the invention the thrombomodulin analogue has a modification of the phenylalanine residue at position 389 (SEQ ID NO:1 or SEQ ID NO:3). The phenylalanine residue is preferably substituted with the following amino acids, ordered in decreasing cofactor activity of the resulting mutant Phe389X-TM analogue (see FIG. 8C): Val, Glu, Thr, Ala, His, Trp, Asp, Gln, Leu, Ile, Asn, Ser, Arg, Lys, Met, Tyr, Gly, Cys, Pro.

In another embodiment of the invention the interdomain loop of the TM consisting of the three amino acids Gln³⁸⁷, Met³⁸⁸ and Phe³⁸⁹ is partially or completely deleted or inserted by one or more amino acids, preferably by an alanine residue (see FIG. 8D).

For these preferred TM analogues with modifications at positions Phe³⁷⁶, Gln³⁸⁷, Met³⁸⁸ or Phe³⁸⁹, the TM analogue can be a full length or a soluble TM analogue, comprising the EGF domains EGF1 to EGF6, preferably comprising the EGF domains EGF3 to EGF6. In a preferred embodiment these analogues contain the substitutions that are given in the TM analogue Solulin. In a more preferred embodiment these Solulin-derived TM analogues consist only of EGF1 to EGF6, in particular of the EGF domains EGF3 to EGF6.

In an embodiment of the invention the thrombomodulin analogue is used in its oxidised form. Several techniques are known to the skilled person for a controlled oxidation of proteins. The TM analogue is preferably oxidised using chloramine T, hydrogen peroxide or sodium periodate.

The invention further pertains to a method that is useful for screening TM analogues to be used for the treatment of coagulopathy with hyperfibrinolysis. This method comprises a first step of modifying the amino acid sequence of thrombomodulin by insertion, deletion or substitution of one or more amino acids, preferably in the EGF domains EGF1 to EGF6, more preferably in the EGF domains EGF3 to EGF6, and most preferably between the amino acid positions Asp³⁴⁹ and Asp⁴⁶¹. For the person skilled in the art several techniques are known to modify protein sequences e.g. by site-directed mutagenesis or random mutagenesis with subsequent selection.

In a second step the modified TM analogue is compared with a control protein for one or more of the following characteristics selected from the group consisting of: binding affinity to thrombin (K_{D} value), cofactor activity, TAFI activation activity or TAFIa potential, ratio of TAFI activation activity and cofactor activity, effect of protein oxidation, effect on clot lysis in time in an in vitro assay, or the effect in a coagulation-associated animal model.

As a control protein, a thrombomodulin protein or analogue is used, preferably a rabbit lung thrombomodulin or a human TM analogue comprising the six EGF domains. The TM analogue can have the native amino acid sequence or alternatively can possess one or more modifications such as the M388L substitution.

The invention further relates to a method of treating coagulopathy with hyperfibrinolysis, comprising the administration of a therapeutically effective amount of a thrombomodulin analogue exhibiting an antifibrinolytic effect.

Particularly this method of treatment comprises TM analogues exhibiting one or more of the following features in comparison with a control protein: a decreased binding affinity towards thrombin, a binding affinity towards thrombin with a k_{D} value of more than 0.2 nM, a significantly reduced cofactor activity, or an increased ratio of TAFI activation activity to cofactor activity. As a control protein, a thrombomodulin protein or analogue is used, preferably a rabbit lung thrombomodulin or a human TM analogue comprising the six EGF domains. The TM analogue can have the native amino acid sequence or alternatively can possess one or more modifications such as the M388L substitution.

### DEFINITIONS

As used in the context of the present invention the term “antifibrinolytic effect” shall refer to the ability of a thrombomodulin analogue to prolong the clot lysis time (as described in Example I) compared to identical assay conditions without addition of the thrombomodulin analogue. The antifibrinolytic effect is due to a prevalence of the antifibrinolytic activity of the TM analogue compared to its profibrinolytic activity.

As used herein the term “profibrinolytic effect” shall refer to the ability of a thrombomodulin analogue to significantly reduce the clot lysis time in an in vitro assay (as described in Example I) compared to identical assay conditions without addition of the thrombomodulin analogue.

The terms “significantly reduce” and “significantly prolong” as used herein refers to a prolongation or reduction of the clot lysis time that is significantly different from the basis value at the p=0.1 level and/or refers to a prolongation or reduction that exceeds 10%, preferably 20%, more preferably 30% and most preferably 40%, 50%, 60%, 70%, 80% 100%, 150% or 200%.

As used in the context of the present invention the words “treat,” “treating” or “treatment” refer to using the TM analogues of the present invention or any composition comprising them to either prophylactically prevent a bleeding event, or to mitigate, ameliorate or stop a bleeding event. They encompass either curing or healing as well as mitigation, remission or prevention, unless otherwise explicitly mentioned. Also, as used herein, the word “patient” refers to a mammal, including a human.

As used herein the term “coagulopathy with hyperfibrinolysis” shall refer to a coagulopathy as a disease affecting the coagulability of the blood, whereby a markedly increased fibrinolysis causes, aggravates or prolongs bleeding events.

As used in the context of the present invention the term “thrombomodulin analogue” refers to both protein and peptides having the same characteristic biological activity as membrane-bound or soluble thrombomodulin. Biological activity is the ability to act as a receptor for thrombin and increase the activation of TAFI, or other biological activity associated with native thrombomodulin.

The term “binding affinity” used herein refers to the strength of the affinity between the thrombomodulin analogue and thrombin and is described by the dissociation constant K_{D}. The K_{D} value for the binding affinity between thrombin and thrombomodulin may be determined by equilibrium methods, (e.g. enzyme-linked immunoabsorbent assay (ELISA) or radioimmunoassay (RIA)) or kinetics (e.g. BIACORE™ analysis), for example. The binding affinity is preferably analysed using a kinetics assay as described in Example II of the present invention.

“K_{D}” refers to the relative binding affinity between the TM analogue and thrombin. High K_{D} values represent low binding affinity. The precise assays and means for determining K_{D} are provided in example II.

The term “cofactor activity” as used herein refers to the ability of the thrombomodulin analogues to complex with thrombin and potentiate the ability of thrombin to activate protein C. The assay procedures used to measure cofactor activity are given in Example III of the present invention.

The terms “TAFI activation activity” as used herein refers to the ability of the thrombomodulin analogues to complex with thrombin and potentiate the ability of thrombin to activate TAFI. The assay procedures used to measure TAFI activity is given in Example IV of the present invention.

“Km” refers to the Michaelis constant and is derived in the standard way by measuring the rates of catalysis measured at different substrate concentrations. It is equal to the substrate concentration at which the reaction rate is half of its maximal value. The “Km” for the TM analogues of the present invention is determined by keeping thrombin concentrations at a constant level (e.g. 1 nM) and using saturation levels of TM (e.g. 100 nM or greater) depending on the K_{D}. Reactions are carried out using increasing concentrations of protein C (e.g., 1-60 μM). Km and kcat are then determined using Lineweaver-Burke plotting or nonlinear regression analysis.

“TM_{E}” refers to an analogue of TM consisting of the six EFG repeats (amino acids 227 to 462 according to SEQ ID NO:1 or SEQ ID NO:3).

“TM_{E}M388L” refers to an analogue of TM consisting of the six EFG repeats (aa 227 to 462) with a substitution of the native methionine at position 388 (based on SEQ ID NO:3) by an leucine residue.

The term “therapeutically effective amount” is defined as the amount of active ingredient that will reduce the symptoms associated with coagulopathy with hyperfibrinolysis, such as bleeding events. “Therapeutically effective” also refers to any improvement in disorder severity, frequency or duration of incidence compared to no treatment.

### EXAMPLE

### Clot Lysis Assay in Human Plasma

Using a model of in vitro clot lysis the ability of soluble thrombomodulin (Solulin) to decrease or increase the clot lysis time in mixtures of normal plasma and Factor VIII deficient plasma was tested.

### Test System

Within the plasma compositions the clotting was initiated in vitro by mixing thrombin (Factor IIa), calcium chloride and phosphatidylcholine/phosphatidylserine (PCPS) vesicles. Time course of coagulation and fibrinolysis were determined with a turbidity assay, and the “TAFIa potential” using a functional assay.

### Experimental Procedures

Materials.

Thrombin and fibrinogen were prepared as described in Walker et al. (J. Biol. Chem. 1999; 274: 5201-5212) with one exception: for the fibrinogen preparation, the solution was made to 1.2% PEG-8000 instead of 2% PEG-8000 by the addition of 40% (w/v) PEG-8000 in water, subsequent to β-alanine precipitation. This change in protocol allowed for a greater yield of fibrinogen. QSY-FDPs (fibrin degradation products that are covalently attached to the quencher, QSY9 C5-maleimide) and TAFIa standards used in the TAFIa assay were prepared as described (Kim et al., 2008; Anal. Biochem 372: 32-40; Neill et al., 2004; Anal. Biochem. 330: 332-341) and recombinant human Pg (S741C) and the fluorescein derivative (5IAF-Pg) were prepared as described by Horrevoets et al. (J. Biol. Chem 1997; 272: 2176-2182). S525C-prothrombin was purified and fluorescently labelled with 5-iodoamidofluorescein (5IAF) as previously described by Brufatto et al. (J. Biol. Chem. 2001; 276: 17663-17671). QSY9 C5-maleimide and 5-iodoamidofluorescein were purchased from Invitrogen Canada Inc. (Burlington, ON, Canada). Plasmin was purchased from Haematologic Technologies Inc. (Essex Junction, Vt., USA) and recombinant human soluble thrombomodulin (Solulin; sTM) was provided from Paion Deutschland GmbH (Aachen, Germany). Normal human pooled plasma (NP) was obtained from healthy donors at the blood bank in the Kingston General Hospital (KGH) in Kingston, Ontario, Canada, and FVIII-deficient plasma (FVIII-DP) was purchased from Affinity Biologicals, Inc. (Hamilton, ON, Canada). TAFI-deficient plasma (TDP) was prepared by affinity chromatography of normal plasma on a column of immobilized anti-human TAFI monoclonal antibody, as described by Schneider et al., (J. Biol. Chem. 2002; 277: 1021-1030). The plasmin inhibitor D-Val-Phe-Lys chloromethyl ketone (VFKck), the thrombin inhibitor D-Phe-Pro-Arg chloromethyl ketone (PPAck) and potato tuber carboxypeptidase inhibitor (PTCI) were purchased from Calbiochem (San Diego, Calif., USA). Tissue-type plasminogen activator (Activase; tPA) was purchased from the pharmacy at KGH (Kingston, ON, Canada). All other reagents were of analytical quality.

### Methods.

### Clot Lysis Assays and the Preparation of Samples to Determine the Extent of TAFI Activation.

FVIII-DP was mixed with NP so that the final percentage of NP was 0, 1, 6, 10, 50 or 100% (0-100% NP). Before mixing, each plasma was diluted to an optical density of 32 and added to an equal volume of a solution containing 1.5 nM tPA, 40 μM PCPS and 20 mM CaCl₂ in the presence or absence of 20 nM thrombin (final concentrations: 0.75 nM tPA, 20 μM PCPS, 10 mM CaCl₂, ±10 nM thrombin) and the samples were divided into multiple Eppendorf tubes and placed in a 37° C. water bath. Clotting and lysis were stopped in these tubes at various time points by the addition of 10 μM PPAck and 10 μM VFKck to selectively inhibit thrombin and plasmin, respectively. The samples were mixed vigorously, then centrifuged for 30 s at 16 000 g (room temperature) and immediately placed on ice to prevent thermal inactivation of TAFIa. The supernatant of each sample was serially diluted by 5-fold with TAFI-deficient plasma and TAFIa was measured using a functional assay described by Kim et al. (Anal. Biochem 2008; 372: 32-40). Identical experiments were conducted in a covered, 96-well plate and the turbidity was monitored at 400 nm over time using a SpectraMax Plus spectrophotometer (Molecular Devices, Sunnyvale, Calif., USA) to determine the timing of coagulation and fibrinolysis. Similar experiments were conducted in the presence or absence of soluble thrombomodulin (0-100 nM) at 4 tPA concentrations (0.25, 0.75, 1.5 and 3 nM) to determine the effect of sTM on TAFI activation and lysis times. These experiments were also conducted in the presence of 5 μM PTCI to show the TAFIa dependent prolongation of lysis in normal and FVIII-deficient plasma.

### Determination of the Time Course of Prothrombin Activation in Normal and FVIII-Deficient Plasma.

Normal and FVIII-deficient plasmas (0-100% NP) were supplemented with the prothrombin derivative (5IAF-II; 300 nM final) as well as 20 μM PCPS and 10 mM CaCl₂ in the presence of 10 nM thrombin to initiate clotting. These experiments were conducted in an opaque, plastic-covered 96-well plate. A SpectraMax GeminiXS (Molecular Devices, Sunnyvale, Calif., USA) was used to monitor fluorescence intensities over time at 37° C. with excitation and emission wavelengths of 495 nm and 535 nm, respectively, employing a 530-nm emission cut-off filter. Fluorescence was normalized (0-1) to reflect the baseline and maximal fluorescence, which correlates with full prothrombin activation.

### Determination of the TAFIa Potential.

The area under the TAFIa plots was chosen as a parameter to quantify the effect of TAFIa over the course of the experiments. This parameter was designated the “TAFIa potential” by analogy with the “thrombin potential” defined by Hemker et al. (Thromb. Haemost. 1993; 85: 5-11). TAFIa potential, like thrombin potential, is proportional to the amount of substrate cleaved and is explained mathematically, as follows:

$$\frac{d S}{d t} = - \frac{{k}_{cat}}{{K}_{m}} [ TAFIa ] [ S ] , \qquad ( 1 )$$

where dS/dt is the rate of substrate consumption and S is the substrate.
If S is constant (i.e. limited consumption of S),

$$\begin{matrix}\frac{d S}{d t} = - S \frac{{k}_{cat}}{{K}_{m}} [ TAFIa ] & ( 2 ) \\ d S = - S \frac{{k}_{cat}}{{K}_{m}} [ TAFIa ] d t \\ For some interval 0 to t , & ( 3 ) \\ {\int }_{S ( 0 )}^{S ( t )} d S = - S \frac{{k}_{cat}}{{K}_{m}} {\int }_{0}^{t} [ TAFIa ] d t & ( 4 )\end{matrix}$$

Realizing that the integral on the right in equation (4) is the area under the TAFIa plot,

$$\begin{matrix}Δ S ( t ) = - S \frac{{k}_{cat}}{{K}_{m}} ( area under curve ) & ( 5 ) \\ Δ S ( t ) = - S \frac{{k}_{cat}}{{K}_{m}} ( TAFIa Potential ) & ( 6 )\end{matrix}$$

### Results

### Clot Lysis Time is Increased by Addition of Normal Plasma to FVIII-Deficient Plasma.

Clotting was initiated with 10 nM Factor IIa, 10 mM CaCl₂ and 20 μM PCPS vesicles to create a model where the clot structure is insensitive to the FVIII concentration. Because the clot structure is similar regardless of the FVIII concentration, the effect of FVIII on tPA-dependent (0.75 nM) clot lysis can be determined. Using this lysis model, lysis times increased as the percentage of normal-plasma increased. FIG. 1 shows the clot lysis profiles for FVIII-DP with 0-100% added normal plasma and the lysis times are summarized in FIG. 1 (inset). In FVIII-DP the lysis time is 37 min and can be increased by approximately 50% by the addition of normal plasma.

### 10% Normal Plasma is Sufficient to Restore Clot Lysis in FVIII-DP.

At 10% normal plasma the shortened lysis time associated with FVIII-DP has been corrected to that observed in normal plasma (see FIG. 1, inset).

### 50% of the TAFIa Potential is Sufficient to Restore Clot Lysis in FVIII-DP.

TAR activation was measured in normal, FVIII-deficient and mixed plasmas to quantify the effect of FVIII on the time course of activation. A functional assay was used to measure TAFIa over the time course of clotting and lysis and the results are presented in FIG. 2. When thrombin, calcium ions and PCPS were used to initiate clotting in FVIII-DP, approximately 30 pM TAFIa was measured after 5 min. As the percentage of normal plasma increased so too did the peak concentration of TAFIa. Although the lysis time was corrected by supplementing FVIII-DP with 10% normal plasma, this was not sufficient to fully correct TAFI activation. By calculating the area under the TAFIa time course plots (FIG. 2A) it was determined that approximately the same TAFIa potential (FIG. 2B) was achieved over the first 50 min in normal plasma and 50% normal plasma (16 800 pM mins and 14 100 pM mins, respectively) but FVIII-DP plasma mixed with 10% normal plasma had a TAFIa potential of only 50% of the TAFIa potential in normal plasma.

### There is a Strong Correlation Between Lysis Time and TAFIa Potential.

In order to quantify the relationship between lysis time and TAFI activation over the range 0-100% FVIII, log lysis time vs. log TAFIa potential was plotted (FIG. 2B, inset). As expected, the data show a strong positive correlation between lysis time and TAFIa potential in plasma containing 0-100% FVIII. The TAFI activation profile in FIG. 2A can be rationalized by analyzing prothrombin activation in plasma (FIG. 3) because thrombin is the activator of TAFI. The general trend is that as the percentage of normal plasma increased, the rate of prothrombin activation also increased (which can be determined by examining the slope of the curve in FIG. 3). An exception occurs with normal plasma. In normal plasma the rate of prothrombin activation is lower than in FVIII-DP mixed with 50% normal plasma. While the rate is slower in normal plasma, prothrombin activation persists for about twice as long as in FVIII-DP mixed with 50% normal plasma. In every experiment, the timing of prothrombin activation corresponds well with TAFI activation. Normal plasma was also clotted using calcium ion and PCPS, without added thrombin. Calcium-induced coagulation does not occur immediately; it takes approximately 15 min for the clot to form in normal plasma. At this time, prothrombin activation enters the propagation phase and as a result, TAFI is activated. The extent and timing of TAFI activation with respect to clot formation is the same whether clotting is initiated in the presence or absence of added thrombin, which suggests that TAFI activation is a result of thrombin generated in situ and not of thrombin added to induce clotting. In the presence of thrombin there was a TAFIa potential of 16,800 pM min compared with 14,150 pM min in the absence of thrombin.

### Soluble Thrombomodulin Prolongs Clot Lysis in Normal and FVIII-Deficient Plasma.

In normal plasma, peak TAFIa levels and TAFIa potential increased from 600 pM and 16 800 pM min, respectively, in the absence of sTM to approximately 6000 pM and 150,000 pM min, respectively, in the presence of 10 nM sTM. This increase in TAFI activation resulted in a 70% increase in the lysis time. The effect of 10 nM sTM on the relative prolongation of lysis in FVIII-DP was similar to normal plasma in that lysis was prolonged by 65% when FVIII-DP was clotted and lysed in the presence of sTM. In the presence of 10 nM sTM, 750 pM TAFIa was present at peak TAFIa concentration compared with 30 pM in the absence of sTM. In the time from clot initiation to the clot lysis time the TAFIa potential was measured to be 12 800 pM min in the presence of 10 nM sTM compared with 600 pM min in the absence of sTM.

### The Increase of Clot Lysis Time in Normal and FVIII-Deficient Plasma Depends on tPA and sTM Concentrations.

The effect of TAFI activation on lysis time was analyzed over a range of tPA and sTM concentrations to determine if the lysis defect in FVIII-DP could be corrected by stimulating TAFI activation. The lysis times summarized in FIG. 4 are relative to lysis times from similar experiments containing PTCI, which is an inhibitor of TAFIa. In the presence of PTCI, there is no functional TAFIa so the relative lysis times presented in FIG. 4 are representative of TAFIa-dependent prolongation of lysis. At the lowest concentration of tPA (0.25 nM), the maximal TAFIa-dependent prolongation of lysis (2-fold) was observed when 1 nM sTM was added to normal plasma. Supplementing FVIII-DP with sTM caused a dose-dependent prolongation of the lysis time (FIG. 4). When 100 nM sTM was added to FVIII-DP the lysis time was fully corrected to that seen in normal plasma. As the tPA concentration increased, a higher concentration of sTM was required to get maximal TAFIa-dependent prolongation of lysis. For example, when 1.5 nM tPA (FIG. 4) is present, 25 nM sTM is required to maximize the TAFIa dependent prolongation of lysis in normal plasma and 100 nM sTM is required in FVIII-DP. Also, as tPA is increased in these clot lysis experiments TAFIa appears to have a much greater effect on lysis time (up to 5.2-fold at 1.5 nM tPA compared with 2.3-fold at 0.25 nM tPA). It appears that as the tPA concentration is increased, the concentration of sTM required to get any TAFIa-dependent prolongation of lysis also increases. At 0.25 nM tPA, no sTM was required to get prolongation of lysis in normal plasma whereas 25 nM sTM was required to get prolongation of lysis when 3 nM tPA (FIG. 4) was added to normal plasma. In order to show how the actual lysis times are affected by tPA and sTM the lysis times in TAFIa inhibited normal and FVIII-deficient plasma are presented in Table 1.

### Thrombomodulin Very Substantially Promotes TAFI Activation and Prolongs Lysis in Both Normal and FVIII-Deficient Plasma.

In normal plasma TAFI activation is shown to be significantly increased in the presence of 10 nM sTM (•; 6000 pM TAFIa at its peak level) compared to the absence of sTM (∘; 600 pM TAFIa; see FIG. 5 A). The accompanying clot-lysis profile reveals that the addition of 10 nM sTM resulted in a 70% increase in the lysis time. In FVIII-DP supplemented with 10 nM sTM TAFIa was measured to be 750 pM at its peak compared to 30 pM in the absence of sTM (see FIG. 5 B). The increase in TAFI activation resulted in a 60% prolongation of lysis compared to FVIII-DP lacking sTM.

### II. Example

### Analysis of Binding Affinity Between Thrombin and Thrombomodulin

Using a fluorescent kinetics assay the affinity expressed as a K_{D} value was determined for the binding between thrombin and the thrombomodulin analogue.

### Test System

The affinity for the binding between thrombin and the thrombomodulin analogue was determined using a fluorescent kinetics assay and expressed as a K_{D} value.

### Experimental Procedures

Materials.

The human thrombin was isolated from plasma as described by Bajzar et al. (J. Biol. Chem. 1995; 270: 14477-14484). Recombinant soluble thrombomodulin (Solulin) was obtained from PAION Deutschland GmbH (Aachen, Germany). All other reagents were obtained from Sigma in analytical quality.

### Methods.

### Measurement of the Binding of Thrombin to Thrombomodulin and TAFI

The binding of thrombin to thrombomodulin was measured as an equilibrium binding assay. A solution containing thrombin (20 nM), thrombomodulin (1.54 μM), and DAPA (20 nM, dansylarginine N-3-(ethyl-1,5-pentanediyl)amide, a fluorescent, reversible thrombin inhibitor) in 0.02 M Tris-HCl, 0.15 M NaCl, 5.0 mM CaCl₂, 0.01% Tween 80, pH 7.4, was added in small successive aliquots to an otherwise identical solution that lacked thrombomodulin. The additions were performed in a cuvette fitted with a magnetic stirrer in the sample compartment of a Perkin-Elmer model LS50B spectrofluorimeter. Intensity values were continuously recorded with excitation and emission wavelengths of 280 and 545 nm, respectively. A 430-nm cut-off filter was used in the emission beam. The data were analyzed as follows. The intensity of fluorescence, I, was assumed to be the sum of intensities from thrombin-DAPA (T·D) and thrombin-thrombomodulin-DAPA (T·TM·D). That is, I=i₁·[T·D]+i₂·[T·TM·D], where and i₁ and i₂ are the coefficients of fluorescence for T·D and T·TM·D (since excitation was at 280 nm, the emission from free DAPA was negligible). Because TM does not appreciably alter the Kₘ for either protein C activation or TAFI activation (see Bajzar et al., 1996; J. Biol. Chem. 271: 16603-16608), it can be assumed that it does not alter the affinity of the thrombin-DAPA interaction.

Thus

[T·D]=([T]+[T·D])/(1+K_{DAPA}/[DAPA])

and

[T·TM·D]=([T·TM]+[T·TM·D])/(1+K_{DAPA}/[DAPA]),

where K_{DAPA} is the dissociation constant for the thrombin-DAPA interaction.

Therefore,

I=i₁·([T]+[T·D])/(1+K_{DAPA}/[DAPA])+i₂([T·TM]+[T·TM·D])/(1+K_{DAPA}/[DAPA]).

If f and b are defined as the fractions of thrombin, respectively, free and bound to thrombomodulin, and [T]₀ is the total concentration of thrombin, then f=([T]+[T·D])/[T]₀, b=([T·TM]+[T·TM·D])/[T]₀ and f+b=1. The fluorescence intensity then is given by I=i₁·f[T]₀/(1+K_{DAPA}/[DAPA])+i₂·b[T]₀/(1+K_{DAPA}/[DAPA]). If I₀ is defined as the initial intensity when no thrombomodulin has been added, then f=1 and I₀=i₁[T]₀/(1+K_{DAPA}/[DAPA]). Similarly, if Iₘₐₓ is defined as the intensity upon saturation of thrombin with thrombomodulin, then b=1 and Iₘₐₓ=i₂[T]₀/(1+K_{DAPA}/[DAPA]). Thus, I=I₀·f+Iₘₐₓ·b. Substituting 1−b for f then gives: I=I₀+(Iₘₐₓ−I₀)·b or ΔI=ΔIₘₐₓ·b. Normalizing to the initial intensity gives (ΔI/I₀)=(ΔIₘₐₓ/I₀)·b. If DAPA binds T and T·TM with equal affinity, then TM binds T and T·D with equal affinity.

Therefore, with K_{TM} defined as the dissociation constant for the thrombin-thrombomodulin interaction, [T][TM]=K_{TM}[T·TM]; [T·D][TM]=K_{TM}[T·TM·D]; and ([T]+[T·D])·[TM]=K_{TM}([T·TM]+[T·TM·D]). The last expression is identical to f·[TM]=K_{TM}·b. Since f=1−b and [TM]=[TM]₀−b·[T]₀, where [TM]₀ is the total thrombomodulin concentration, the following equation is obtained: (1−b)([TM]₀−b·[T]₀)=K_{TM}·b. This is a quadratic equation in b, which when solved and substituted in the expression above for (ΔI/I₀) gives the equation: (ΔI/I₀)=(ΔIₘₐₓ/I₀)·0.5·(K_{TM}+[T]₀+[TM]₀−((K_{TM}+[T]₀+[TM]₀)²−4·[T]₀·[TM]₀)^{1/2}). This latter equation expresses the relationship between fluorescence intensity values, the nominal concentrations of thrombomodulin and thrombin, the dissociation constant for the thrombin-thrombomodulin interaction, and the fluorescence intensity increment that signals the interaction of thrombomodulin with thrombin-DAPA. Intensity data were fit to the above equation by nonlinear regression analysis, with [TM]₀ as the independent variable and K_{TM} and ΔIₘₐₓ as best-fit parameters.

### Results

Thrombin Binds to Soluble Thrombomodulin with an Affinity of K_{D}=23±14 nM.

The binding of thrombin to soluble thrombomodulin was measured by perturbation of the fluorescence of DAPA. As depicted in FIG. 6, the titration curve showed a increase of the relative fluorescence for the concentration range of soluble thrombomodulin between 0 and 75 nM. The data analysis revealed that thrombin binding to soluble thrombomodulin was characterized by K_{D}=23±14 nM.

### III. Example

### Analysis of Cofactor Activity for Mutated Thrombomodulin Analogues

Using a fluorescent kinetics assay the affinity expressed as a K_{D} value was determined for the binding between thrombin and the thrombomodulin analogue.

### Experimental Procedures

### Materials and Methods.

The ability of TM mutants to act as cofactor for thrombin-mediated activation of protein C was assayed directly in the shockates. Recombinant human protein C was from Dr. John McPherson, Genzyme Corp., Framingham, Mass., and was purified as described (BioTechnology 1990; 8: 655-661). Twenty five μl of each shockate was mixed with equal volumes of recombinant human protein C (final concentration of 0.3 μM) and human alpha thrombin (Sigma Chemicals, St. Louis, Mo.) at a final concentration of 1 nM in a microtiter plate. All reagents used were diluted in 20 mM Tris, pH7.4/100 mM NaCl/3.75 mM CaCl₂/0.1% NaN₃ (w/V) containing 5 mg/ml bovine serum albumin. Mixtures were incubated for 1 hr at 37° C. and the reaction was terminated by addition of 25 μl of hirudin at 800 units/ml (Sigma Chemicals, St. Louis, Mo.). The amount of activated protein C was determined by addition of 100 μl of chromogenic substrate D-valyl-L-leucyl-L-arginine-p-nitroanilide (S-2266) (1 mM). The change is measured by the absorbance at 405 nm with time using a plate reader. Data is recorded as milliOD unit/min and determined for each sample by measuring the absorbance every 10 seconds for 15 minutes using a Molecular Devices plate reader. All assays contained triplicate shockate samples each of DH5 alpha cells transfected with either pSELECT-1 vector (no TM), pTHR211 (wild type) or pMJM57 (pTHR211 with methionine at 388 altered to leucine), as internal controls. Cofactor activities of TM mutants were expressed as mean of that obtained for pMJM57.

### Statistical Analysis.

Each mutant was assayed for activity at least twice (three times for those mutants for which only two positive clones were isolated), and all the data were included in the determination of the significance of difference using Student t-Test. Coefficient of variation between plates was 16.7% (n=18).

### Western Blot Analysis

E. coli shockates were run in 10% Tris-tricine SDS PAGE under reduced conditions according to the manufacturer's specifications (Novex Inc., San Diego, Calif.). Reduced and alkylated samples were prepared by boiling shockates in sample buffer (62.5 mM Tris, pH6.8, 2% SDS, 10% glycerol, 0.0025% bromophenol blue) containing 10 mM dithiothreitol for 10 minutes, followed by incubation with 50 mM iodoacetamide.

Proteins were transferred to nitrocellulose filter in transfer buffer (192 mM glycine, 25 mM Iris, pH8.3, 20% methanol) at 4° C. The nitrocellulose filter was blocked with a blocking buffer (1% bovine serum albumin in 10 mM Tris, pH7.5, 0.9% NaCl, 0.05% NaN₃), and then incubated with mouse polyclonal antiserum (raised against reduced and alkylated EGF domain of human thrombomodulin) in the blocking buffer. After washing with a washing buffer (10 mM Tris, pH7.5, 0.9% NaCl, 0.05% NaN₃, 0.05% Tween 20), the filter was incubated with biotinylated goat anti-mouse IgG antibodies in the blocking buffer containing 0.05% Tween 20. Proteins were detected using the Vectastain ABC solution (Vector Laboratories, Burlingame, Calif.) and ECL detection system (Amersham Corporation, Arlington Heights, Ill.) according to the manufacturers specifications.

### IV. Example

### Analysis of Thrombomodulin Analogues for TAFI and Protein C Activation

Using a fluorescent kinetics assay the affinity expressed as a K_{D} value was determined for the binding between thrombin and the thrombomodulin analogue.

### Experimental Procedures

### Proteins and Reagents.

Truncated forms of thrombomodulin comprising Solulin (residues 4-490), TM_{E} (residues 227-462), TM_{E}c-loop 3-6 (residues 333-462), and TM_{E}i4-6 (residues 345-362) were prepared as described by Parkinson et al. (Biochem. Biophys. Res. Commun. 1992; 185: 567-576). Sf9 cells were transfected with the TM constructs, and the proteins were isolated from the media by a combination of chromatography procedures utilizing anion exchange, gel filtration, and thrombin affinity. Purity, assessed by SDS-polyacrylamide gel electrophoresis and silver staining, was 95% or greater. Human plasma TAFI was isolated as described by Bajzar et al. (J. Biol. Chem. 1995; 270: 14477-14484). Human protein C and thrombin were prepared as described by Bajzar and Nesheim (J. Biol. Chem. 1993; 268: 8608-8616). The thrombin inhibitor dansylarginine N-(3-ethyl-1,5-pentanediyl)amide (DAPA) was synthesized as described by Nesheim et al. (Biochemistry 1979; 18: 996-1003). Point mutants resulting from alanine scanning were generated from the TM_{E}M388L construct. Proteins were expressed in Escherichia coli. The procedures and preparation of periplasmic extracts have been described by Nagashima et al., (J. Biol. Chem. 1993; 268: 8608-8616). HEPES, the basic carboxypeptidase substrate hippuryl-arginine, cyanuric chloride, and 1,4-dioxane were obtained from Sigma. All other reagents were of analytical quality.

Measurement of the Rates of Protein C and TAFI Activation with Point Mutants of Thrombomodulin Analogues.

For the activation of TAFI, a 20-μl aliquot of each periplasmic extract was preincubated with thrombin (13 nM final) in 20 mM HEPES, pH 7.5, 150 mM NaCl, 5 mM CaCl₂ for 5 min at room temperature. The mixtures were then incubated with purified recombinant TAFI (18 nM final) and a substrate, hippuryl-arginine (1.0 mM final), in a total volume of 60 μl for 60 min. The amount of activated TAFI was quantitated by measuring the hydrolysis of hippuryl-arginine to hippuric acid, followed by conversion of hippuric acid to a chromogen with 80 μl of phosphate buffer (0.2 M, pH 8.3) and 60 μl of 3% cyanuric acid in dioxane (w/v). After thorough mixing, absorbance of the clear supernatant was measured at 382 nm. The amount of thrombin-dependent activation of TAFI was calculated by subtracting the background absorbance produced in the absence of thrombin for each mutant. Activation of protein C by TM_{E}M388L-alanine mutants was assayed as follows.

All samples and reagents were diluted in APC assay diluent (20 mM Tris-HCl, pH7.4, 100 mM NaCl, 2.5 mM CaCl₂, 0.5% BSA). Samples and TM standards (0-1 nM) were incubated for 60 min in 60 μl total volume at 37° C. in a 96-well plate with 0.5 μM protein C and 1 nM thrombin to generate APC before being quenched with 20 μl of hirudin (0.16 U/μl, 570 nM). The amount of APC formed was determined by monitoring the hydrolysis of S-2266 (100 μl of 1 mM) at 1-min intervals at 405 nm using a plate reader (Molecular Devices Corp., Menlo Park, Calif.). 1 U of activity generates 1 pmol of activated protein C/min (37° C.).

All assays contained extracts of DH5a cells transfected with either pSelect-1 vector (no TM_{E}), wild-type TM_{E}(M388), or TM_{E}(M388L) as internal controls. Cofactor activities of TM_{E}(M388L) alanine mutants were expressed as percentages of the activity of TM_{E}(M388L). Each TM mutant was assayed for both protein C and TAFI activation in duplicate using three independent preparations of extracts.

### 2. Results

The results obtained with the TM mutants (FIG. 7) indicate that five out of eighth mutants have a substantially reduced cofactor activation. From these five mutants four mutants show also a concomitant reduced activation activity of TAFI. Only the mutation at F376A resulted in a profound loss in protein C activation, but only in a modest reduction in TAFI activation. Intriguingly, the difference in importance of Phe³⁷⁶ for TAFI and protein C activation suggests the requirements for thrombomodulin structure are more constrained when protein C is the substrate of the thrombin-thrombomodulin complex.

### V. Example

### Analysis of Met-Specific TM Mutants for Protein C Activation with Regard to Oxidation

Using specific methionine mutants of thrombomodulin analogues the role of these residues for cofactor activation also with respect to protein oxidation was analysed using a protein C activation assay.

### Experimental Procedures

### Proteins and Reagents.

Human recombinant protein C was from Genzyme Corp. (Boston, Mass.). Bovine thrombin was from Miles Laboratories Inc. (Dallas, Tex.). D-Val-Leu-L-Arg-p-nitroanilide was prepared as described by Glaser et al. (Prep. Biochem. 11975; 5: 333-348). Human alpha-thrombin (4,000 NIH U/mg), bovine serum albumin (fraction V) and chloramine T were from Sigma Chemical Co. (St. Louis, Mo.).

### Expression of TM_{E} (Sf9).

All procedures were performed at 4° C. The DNA sequence encoding the six EGF-like repeats of TM (amino acids 227-462) was linked to the signal sequence of the insect protease, hypodermin A, and the hybrid gene placed under control of the polyhedron gene promoter in the baculovirus shuttle vector pTMHY101. Recombinant virus was generated using standard techniques. Mutant analogues described were prepared by use of a mutator site-specific mutagenesis kit (Stratagene, Inc., La Jolla, Calif.) and virus was prepared for expression in the baculovirus system by the same methods.

Purification and Oxidation with Chloramine T

Growth media containing secreted mutants of TME (Sf9) was clarified by centrifugation, lyophilized and redisolved in 1:10 volume of 0.2% NEM-Ac, pH 7, and 0.008% Tween 80. Aliquots were treated with either 5 μl of H₂0 or 5 μl of 100 mM chloramine T; incubated for 20 min at room temperature; oxidant removed by dilution; desalted on NAP-5 columns (20 mM Tris-HCl, 0.1 M NaCl, 2.5 mM CaCl₂, 5 mg/ml BSA, pH 7.4; Pharmacia Inc.); and assayed for activation of protein C as follows.

### Measurement of TM Cofactor Activity (APC Assay)

All samples and reagents were diluted in APC assay diluent (20 mM Tris-HCl, pH7.4, 100 mM NaCl, 2.5 mM CaCl₂, 0.5% BSA). Samples and TM standards (0-1 nM) were incubated for 60 min in 60 μl total volume at 37° C. in a 96-well plate with 0.5 μM protein C and 1 nM thrombin to generate APC before being quenched with 20 μl of hirudin (0.16 U/μl, 570 nM). The amount of APC formed was determines by monitoring the hydrolysis of S-2266 (100 μl of 1 mM) at 1-min intervals at 405 nm using a plate reader (Molecular Devices Corp., Menlo Park, Calif.). 1 U of activity generates 1 pmol of activated protein C/min (37° C.).

### 2. Results

### Reduced Cofactor Activity of TM by Oxidation of Met388.

Mutant and wild-type TME (Sf9) were expressed in insect cells, treated with chloramines T, assayed for cofactor activity and the results compared (Table 2). When TM_{E} is treated with an oxidant such as chloramine T it looses approx. 85% of its cofactor activity (see Table 2). Site-specific mutations of Met²⁹¹ and Met³⁸⁸ demonstrate that inactivation of TME (Sf9) is due to oxidation of a single methionine. Derivatives that retain Met³⁸⁸ were inactivated by chloramine T to a similar extent (>80%) whereas the Met388Leu mutant was resistant. Mutants in which Met²⁹¹ is replaced were active but were not resistant to oxidative inactivation.

### VI. Example

Analysis of TM Analogues with Mutations of the Interdomain Loop Between EGF4 and EGF5 (Gln387, Met388, Phe389)

sing specific mutants of thrombomodulin analogues the role of these residues and their oxidation was analysed using a protein C activation assay.

### Experimental Procedures

Plasmid Constructions.

A thrombomodulin fragment consisting of only the EGF-like domains (TM_{E}) was expressed in E. coli as follows, DNA fragment coding for TM_{E} (residues 227-462) of full length TM was obtained by polymerase chain reaction from human genomic DNA using primers 5′-CCGGGATCCTCAACAGTCGGTGCCAATGTGGCG-3′ and 5′-CCGGGATCCTGCAGCGTGGAGAACGGCGGCTGC-3′. This fragment was placed under the control of a β-lactamase promoter and signal sequence in pKT279. An EcoRV-BgIII fragment of the resultant plasmid and a ScaI-SacI fragment of pGEM3zf-containing the f1 origin of replication was then inserted into the pSelect-1 vector at the EcoRV-BamHI and ScaI-SacI site, respectively, to construct E. coli expression plasmid pTHR211. Plasmids coding for TM mutants at position 387, 388, or 389 were constructed using a site-directed mutagenesis procedure described in the altered sites in vitor mutagenesis kits with a single stranded pTHR211DNA template. Each primer of the site-specific mutation was confirmed by restriction analysis.

To measure cofactor activity of the mutants, the individual E. coli cultures expressing mutant proteins were centrifuged, washed, and the cell pellets incubated (10 min, 4° C.) in 20% sucrose, 300 mM Tris-HCl, pH 8.0, 1 mM EDTA, 0.5 mM MgCl₂. Shockates were prepared by centrifugation of cell pellets treated with 0.5 mM MgCl₂ (10 min, 4° C.) and assayed in the APC assay. The data are an average of the results from each of three independent clones.

### Measurement of TM Cofactor Activity (APC Assay)

All samples and reagents were diluted in APC assay diluent (20 mM Tris-HCl, pH7.4, 100 mM NaCl, 2.5 mM CaCl₂, 0.5% BSA). Samples and TM standards (0-1 nM) were incubated for 60 min in 60 μl total volume at 37° C. in a 96-well plate with 0.5 μM protein C and 1 nM thrombin to generate APC before being quenched with 20 μl of hirudin (0.16 U/μl, 570 nM). The amount of APC formed was determined by monitoring the hydrolysis of 5-2266 (100 μl of 1 mM) at 1-min intervals at 405 nm using a plate reader (Molecular Devices Corp., Menlo Park, Calif.). 1 U of activity generates 1 pmol of activated protein C/min (37° C.).

### 2. Results

### Reduced Cofactor Activity of TM by Mutation of the Interloop Domain.

Using the site-directed mutagenesis, TM mutants that have either an altered amino acid, a deletion or an insertion at positions 387, 388, or 389 were expressed (FIG. 8). The cofactor activity of the TM mutants are an average obtained from three independent clones and are expressed as a percentage of the activity found for TME(Sf9)WT. Gel scans on the Western blots were performed using a polyclonal antibody against TM for all new mutants at position 388 and for selected mutants at position 387. These scans gave approximately equivalent amounts of TM, indicating that expression differences cannot account for the observed activity differences. In addition, in an independent substitution at position 387 (FIG. 8A), 388 (FIG. 8B), 389 (FIG. 8C), or insertions and deletions anywhere in the inter-domain loop (FIG. 8D) result in analogues which generally are poorer cofactors in the APC assay then wildtype TM_{E}. Analogues where Gln³⁸⁷ is replaced by Thr, Met or Ala retain >70% cofactor activity, but substitution with Glu reduces this to 58% of control, and all other amino acids result in >50% loss. Only the substitution of Met³⁸⁸ with Leu results in a substantially higher cofactor activity (1.8-fold) than wildtype. All other substitutions of Met³⁸⁸ except Gln and Tyr resulted in >50% loss of cofactor activity. TM cofactor activity is less sensitive to amino acid replacement of Phe³⁸⁹ and nine of the point mutants at this position retain >70% of the activity found in the control. Pro or Cys substitution at any positions reduced the activity to >10% except for Met388Pro which retained 30% activity. Varying the length of the interdomain loop between EGF4 and EGF5 by either deleting individual amino acids or inserting an Ala into each of the four possible positions resulted in mutants with less than 10% of the activity of wild type TM_{E}.

### FIGURE LEGENDS

FIG. 1: Clot-lysis profiles and lysis times of factor VIII deficient plasma (FVIII-DP), normal plasma (NP) and FVIII-DP mixed with NP. Clot lysis profiles are shown for 0 (—), 1 (••••), 6 (---), 10 (-••-••-), 50 (———) and 100% (-•-•-) NP. From the clot-lysis profiles, the lysis time was determined by taking the time at which the clot has been degraded to one half of its highest optical density. In the inset, the lysis times are summarized, with the general trend being an increase in lysis time as the percentage of NP (and consequently amount of FVIII) is increased. The effect of adding NP on lysis time reaches a plateau at 10% NP.

FIG. 2: Thrombin activatable fibrinolysis inhibitor (TAFI) activation in plasma containing various percentages of FVIII: (A) When FVIII-deficient plasma (FVIII-DP) is mixed with normal plasma (NP) TAFI activation is enhanced. In FVIII-DP only 30 pM TAFIa was measured at its peak (•) compared with ˜600 pM TAFIa in 50% NP (□) and 100% NP (Δ). These experiments were conducted in triplicate and the data represent the mean±SE. The TAFIa potential (B), defined here as the area under the time course of activation plot (A) from the time of clot initiation to the last time point, increases as the percentage of NP increases to a plateau at 50% NP. The TAFIa potentials of 50% NP and 100% NP are similar (14,100 pM min and 16,800 pM min, respectively) despite the shape of their respective TAFI activation plots being quite different. The relationship between lysis time (FIG. 1, inset) and TAFIa potential, as it relates to FVIII levels, is presented (FIG. 2B, inset) using a plot of log lysis time vs. log TAFIa potential. As expected, the data show a strong positive correlation between lysis time and TAFIa potential in plasma containing 0-100% FVIII.

FIG. 3: Prothrombin activation in plasma containing various percentages of FVIII. The time course of prothrombin activation is shown for FVIIIDP mixed with 0 (•), 1 (▪), 6 (▴), 10 (◯), 50 (□) and 100% NP (Δ). Generally, the rate of prothrombin activation increases as the percentage of NP increases. At 50% NP prothrombin activation occurs at a high rate (as determined by examining the slope of each plot) and appears to be over within 15 min, whereas 100% NP has a slower rate of prothrombin activation over a longer time period.

FIG. 4: The effect of sTM on thrombin activatable fibrinolysis inhibitor (TAFI) activation in normal plasma (NP) and factor VIII deficient plasma (FVIII-DP) at various concentrations of both sTM (0-100 nM) and tPA (0.25-3 nM) The TAFIa-dependent defect in prolonging lysis in FVIII-DP is corrected by the addition of 100 nM sTM to plasma containing 0.25 nM tPA. As the concentration of tPA is increased only partial correction of the lysis defect is observed in FVIII-DP in the presence of 100 nM sTM. In these experiments, potato tuber carboxypeptidase inhibitor (PTCI) was used to create a condition in which there is no functional TAFIa. Therefore, any increase in lysis, as presented by the ratio lysis time/lysis time+PTCI is TAFIa dependent.

FIG. 5: TAFI activation and clot lysis profiles in normal plasma (NP) (A) and FVIII-deficient plasma (FVIII-DP) (B) in the presence of 10 nM thrombomodulin (•) or without thrombomodulin (∘). The accompanying clot-lysis profile is shown (—) and the clot lysis profile for no sTM is shown as a reference (—). These experiments were conducted in triplicate and the data represents the mean±SE.

FIG. 6: Thrombin binding to thrombomodulin. Binding of thrombin to thrombomodulin was determined by titrating 1.5 ml of a solution composed of thrombin (20 nM) and DAPA (20 nM) in 20 mM Tris.HCl, 150 mM NaCl, 5.0 mM Ca²⁺, 0.01% Tween 80 with 1.54 μM thrombomodulin in an identical solution. Fluorescence intensity was measured (λₑₓ=280 nm, λₑₘ=545 nm).

FIG. 7: Relative cofactor activities of point mutants in TAFI and protein C activation. Alanine-scanning mutagenesis was used to prepare point mutations in soluble thrombomodulin. Rates of protein C and TAFI activation (relative to the rate of activation with mutant TM_{E}M388L) are shown for TAFI (solid bars) and protein C (hatched bars).

FIG. 8: Mutations of the interdomain loop between EGF4 and EGF5. Three independent plasmids were constructed in E. coli for each mutant. Shockates were prepared, assayed for cofactor activity by the APC assay, and samples were analysed on Western blots (not shown). Activity values are the average from three separate clones. Panel A, substitution mutants at Gln³⁸⁷; panel B, substitution at Met386; panel C, substitution mutants at Phe389; panel D, deletions and alanine insertions in the interdomain loop. The activity measured for shockates from E. coli transfected with the control plasmid, pSelect, lacking the TM insert is shown. See Clarke et al. (J. Biol. Chem. 1993; 268:6309-6315) for additional details.

FIG. 9: Schematic diagram of the pro- and antifibrinolytic effects of thrombomodulin (modified after Mosnier and Bouma, Arterioscler. Thomb. Vasc. Biol. 2006; 26: 2445-2453). The increase in clot lysis time at low TM concentrations is attributable to stimulation of TAFI activation and illustrates the antifibrinolytic activity of TM. At higher concentrations of the rabbit lung TM the clot lysis time decrease because of the activation of protein C and inhibition of TAFI activation; illustrating the profibrinolytic activity of rabbit lung TM (solid line). Note that above 15 nM the profibrinolytic activity of rabbit lung TM exceeds the antifibrinolytic activity resulting in an overall profibrinolytic effect. In contrast the soluble TM analogue shows only an antifibrinolytic effect (dashed line).

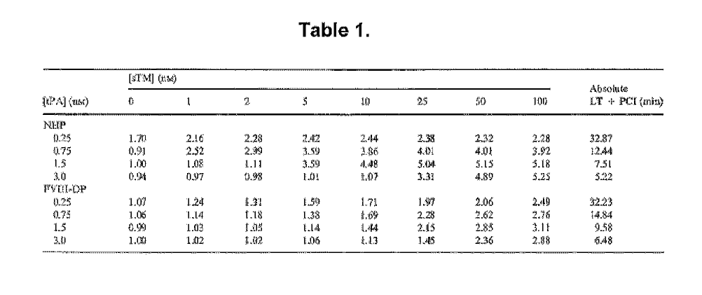

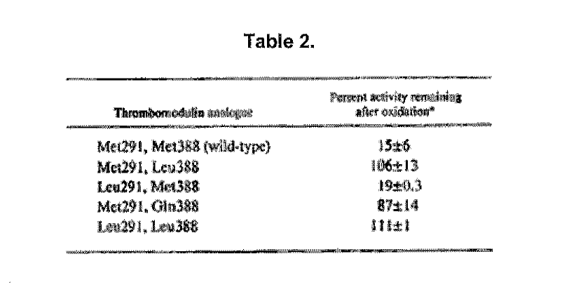

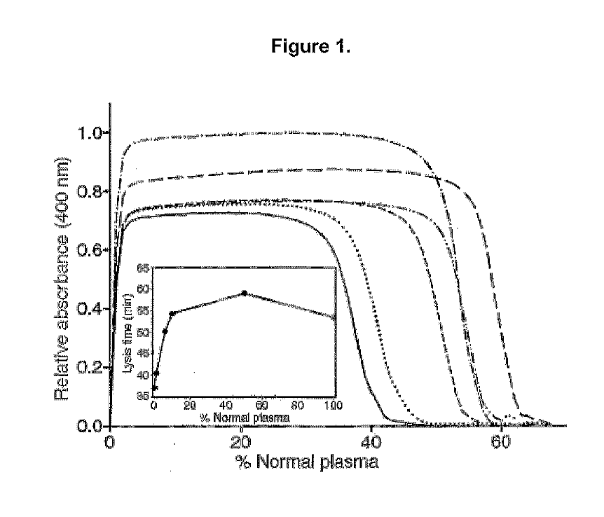

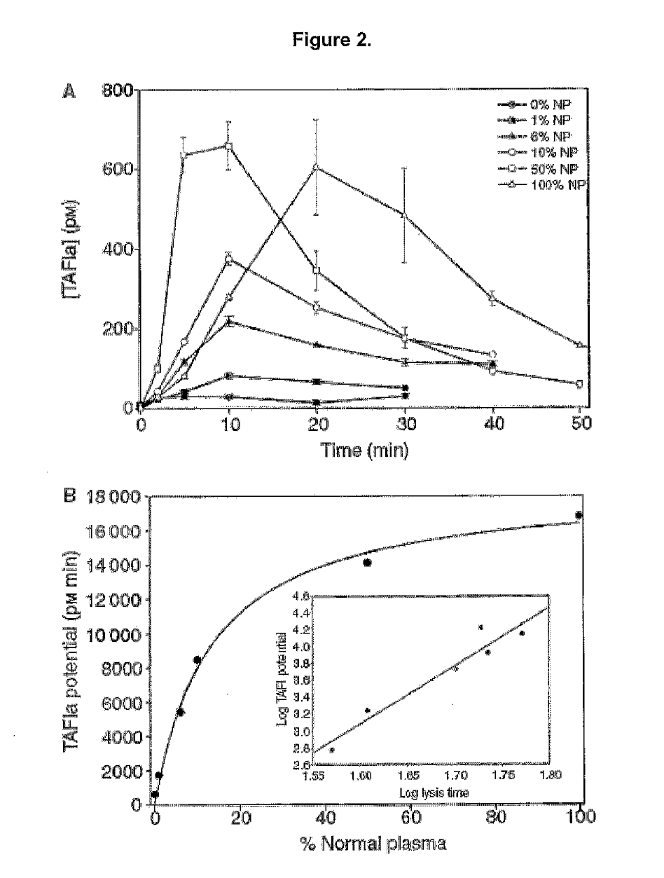

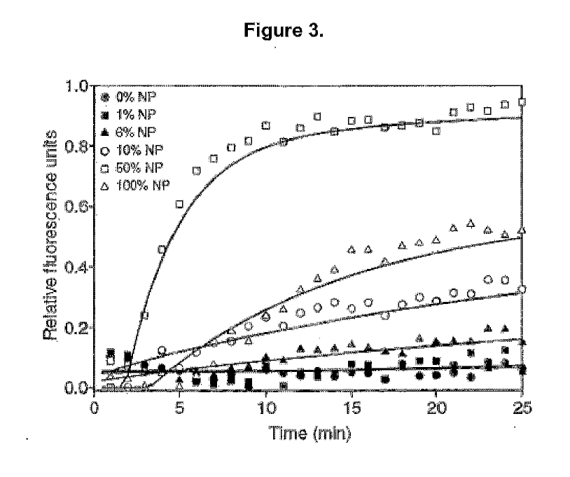

### TABLE LEGENDS

Table 1:

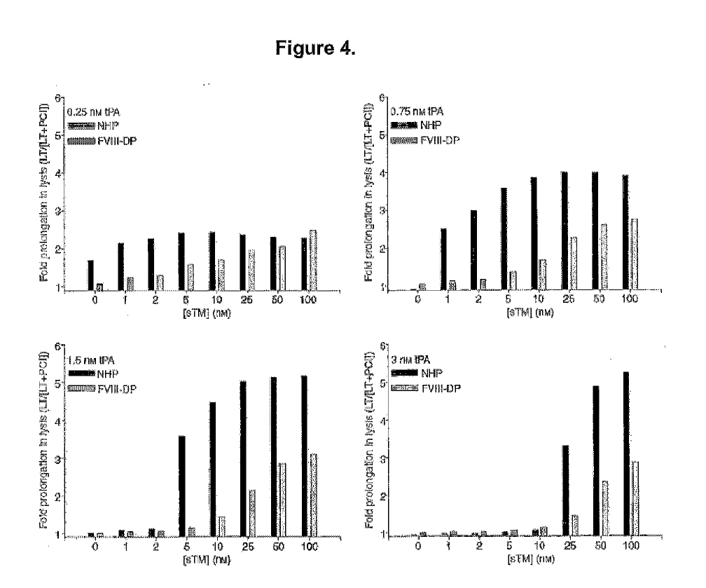

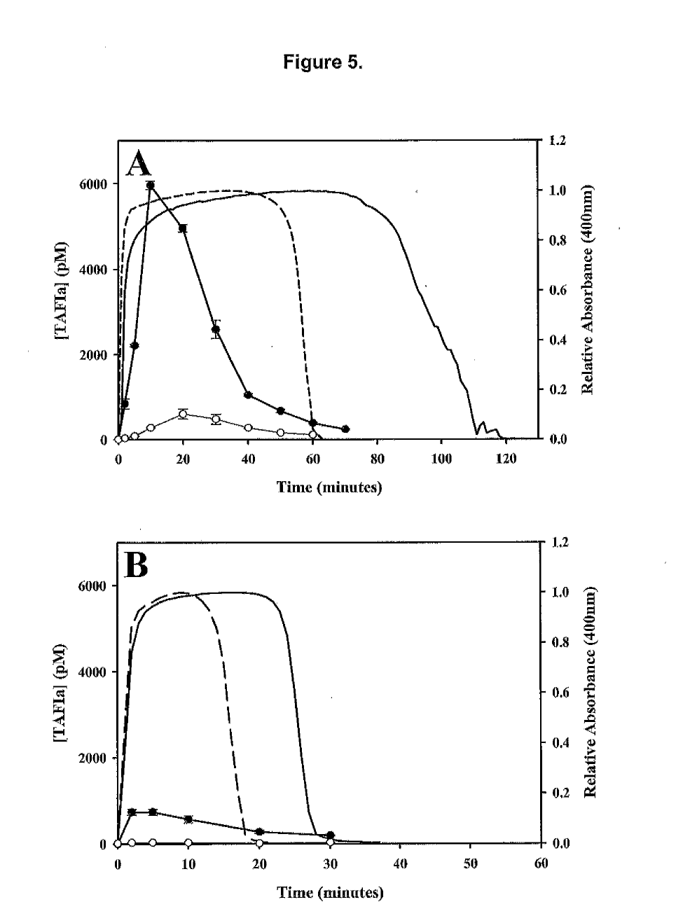

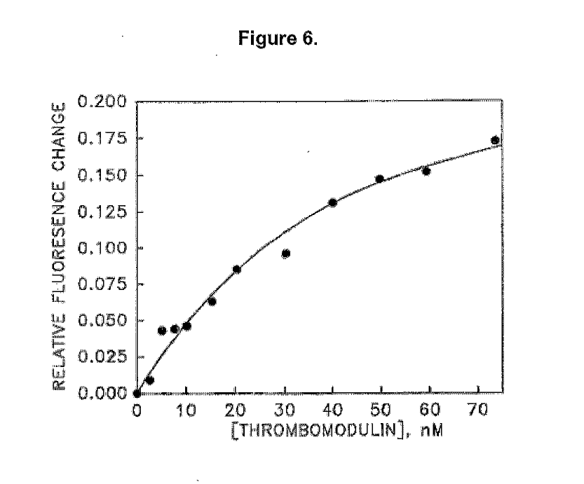

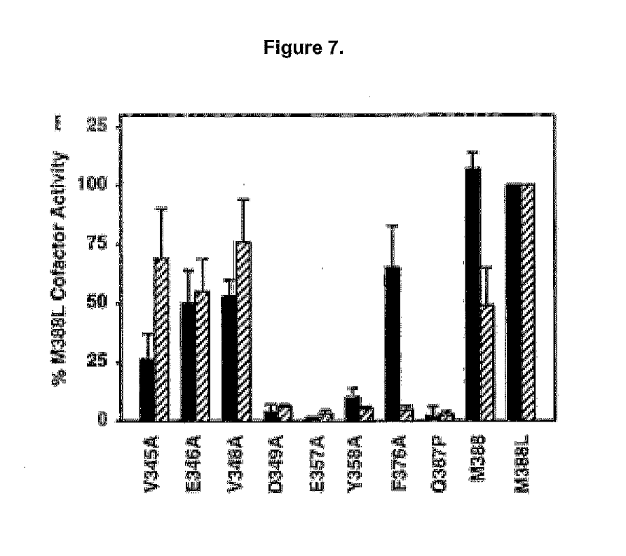

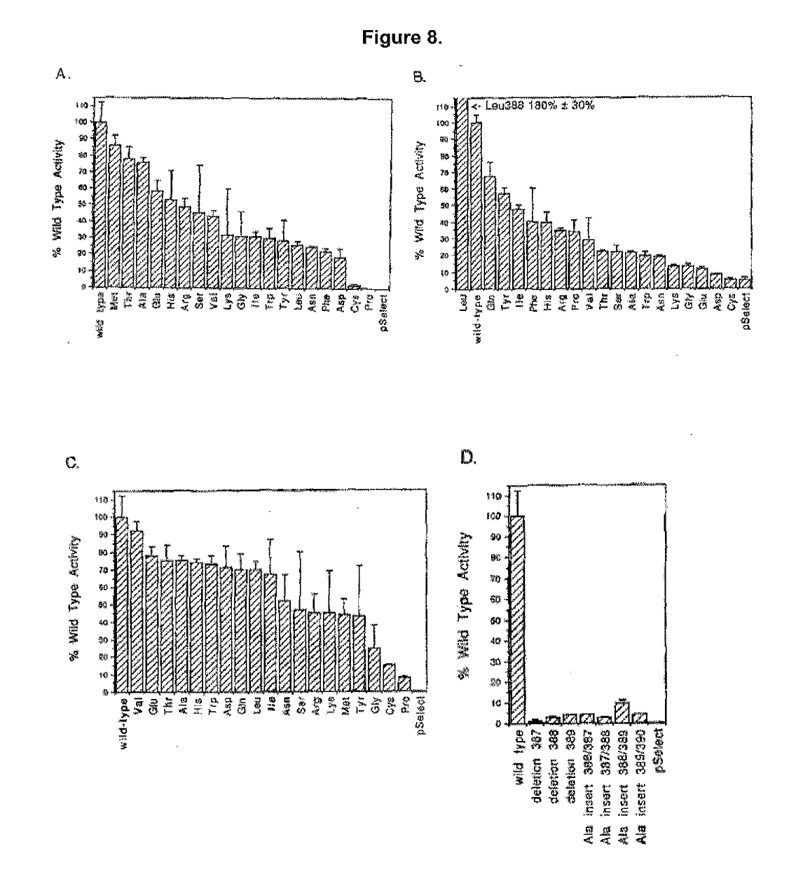

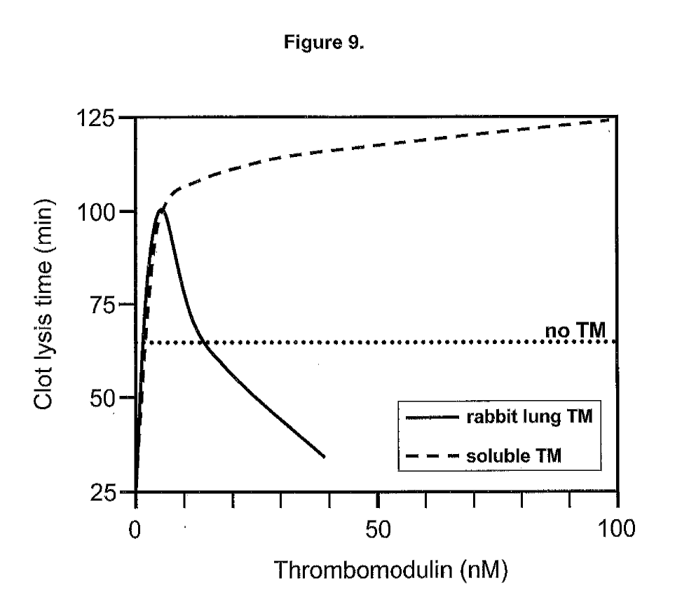

Summary of the data used to construct FIG. 4, including the absolute lysis time in the presence of PTCI to enable determination of the lysis time under each condition. In all cases, the lysis time is expressed relative to that obtained in the presence of the TAFIa inhibitor, PTCI. TAFI, thrombin activatable fibrinolysis inhibitor; PTCI, potato tuber carboxypeptidase inhibitor.

Table 2: Chloramine T Oxidation of Site-Specific Mutant Analogues of TM_{E} (Sf9)

The results after chloramine T treatment were expressed as a percentage of the activity after control treatment. *Average of duplicate determinations and deviation from the mean.

1.-18. (canceled)

19. A method of producing a medicament for the treatment of coagulopathy with hyperfibrinolysis comprising the steps of
providing a thrombomodulin analogue in a sufficient amount to treat of coagulopathy with hyperfibrinolysis, said thrombolinmodulin analogue is selected from the group consisting of:
(i) a thrombomodulin analogue having an amino acid sequence corresponding to the amino acid sequence of mature thrombomodulin (depicted in SEQ ID NO:1 or SEQ ID NO:3) and comprising one or more of the subsequent modifications:
a) removal of amino acids 1-3
b) M388L
c) R456G
d) H457Q
e) S474A, and terminating at P490;
(ii) a thrombomodulin analogue comprising at least one structural domain selected from the group containing EGF3, EGF4, EGF5, EGF6, preferably comprising the fragment EGF3-EGF6 and more preferably comprising the EGF domains 1-6;
(iii) a thrombomodulin analogue consisting of EGF domains EGF1 to EGF6, and more preferably consists of the EGF domains EGF3 to EGF6; and
(iv) a thrombomodulin analogue having an amino acid sequence which comprises a sequence of at least 85%, or at least 90% or 95% sequence identity with SEQ ID NO: 2, and combining it with a suitable carrier.

20. The method according to claim 19, wherein the thrombomodulin analogue has an amino acid modification at one or more positions corresponding to natural sequence at SEQ ID NO: 1 or SEQ ID NO:3):
aa) ³⁴⁹Asp;
ab) ³⁵⁵Asn;
ac) ³⁵⁷Glu;
ad) ³⁵⁸Tyr;
ae) ³⁵⁹Gln;
af) ³⁶¹Gln;
ag) ³⁶³Leu;
ah) ³⁶⁴Asn;
ai) ³⁶⁸Tyr;
aj) ³⁷¹Val;
ak) ³⁷⁴Glu;
al) ³⁷⁶Phe;
am) ³⁸⁴His;
an) ³⁸⁵Arg;
ba) ³⁸⁷Gln;
bb) ³⁸⁹Phe;
bc) ³⁹⁸Asp;
bd) ⁴⁰⁰Asp;
be) ⁴⁰²Asn;
bf) ⁴⁰³Thr;
bg) ⁴⁰⁸Glu;
bh) ⁴¹¹Glu;
bi) ⁴¹³Tyr;
bj) ⁴¹⁴Ile;
bk) ⁴¹⁵Leu;
bl) ⁴¹⁶Asp;
bm) ⁴¹⁷Asp;
bn) ⁴²⁰Ile;
bo) ⁴²³Asp;
bp) ⁴²⁴Ile;
bq) ⁴²⁵Asp;
br) ⁴²⁶Glu;
ca) ⁴²⁸Glu;
cb) ⁴²⁹Asp;
cc) ⁴³²Phe;
cd) ⁴³⁴Ser;
ce) ⁴³⁶Val;
cf) ⁴³⁸His;
cg) ⁴³⁹Asp;
ch) ⁴⁴⁰Leu;
ci) ⁴⁴³Thr;
cj) ⁴⁴⁴Phe;
ck) ⁴⁴⁵Glu;
cl) ⁴⁵⁶Arg;
cm) ⁴⁵⁸Ile; or
cn) ⁴⁶¹Asp.

21. The method according to claim 19, wherein the thrombomodulin analogue is a soluble thrombomodulin analogue.

22. The method according to claim 21, whereas the thrombomodulin analogue is a human soluble thrombomodulin analogue.

23. The method according to claim 19, wherein the thrombomodulin analogue has a modification of the phenylalanine at position 376 according to SEQ ID NO:1 or SEQ ID NO:3.

24. The method of claim 23, wherein the modification of the phenylalanine at position 376 according to SEQ ID NO:1 or SEQ ID NO:3 is substituted with an aliphatic amino acid.

25. The method of claim 24, wherein the substitution is with glycine, alanine, valine, leucine, or isoleucine.

26. The method of claim 20, wherein the thrombomodulin analogue has a modification of one or more of the following amino acids in SEQ ID NO:1 or SEQ ID NO:3:
a) ³⁸⁷Gln;
b) ³⁸⁸Met;
c) ³⁸⁹Phe,
whereby the amino acids are deleted, inserted by one or more additional amino acids or are substituted.

27. The method according to claim 19, wherein the thrombomodulin analogue is used in its oxidized form, preferably oxidized with chloramine T, hydrogen peroxide or sodium periodate.

28. The method according claim 27, whereas one or more of methionine residues within the thrombomodulin analogue are oxidized.

29. The method of claim 28, wherein the methionine residue is oxidized at position 388 according to SEQ ID NO:1 or SEQ ID NO:3.

30. The method according to claim 19, wherein the thrombomodulin analogue exhibits one or more of the following features:
(i) a binding affinity towards thrombin that is decreased compared to the rabbit lung thrombomodulin, and/or a binding affinity towards thrombin with a k_{D} value of more than 0.2 nM;
and/or
(ii) a reduced cofactor activity compared to cofactor activity of the TM analogue TMEM388L,
(iii) an increased ratio of TAFI activation activity to cofactor activity as compared to the TM analogue TM_{E}M388L.

31. The method of claim 19, wherein the coagulopathy with hyperfibrinolysis is selected from the group consisting of haemophilia A, haemophilia B, haemophilia C, von Willebrandt disease (vWD), acquired von Willebrandt disease, Factor X deficiency, parahemophilia, hereditary disorders of the clotting factors I, II, V, or VII, haemorrhagic disorder due to circulating anticoagulants or acquired coagulation deficiency.

32. A method of treating a subject suffering from coagulopathy with hyperfibrinolysis comprising administering a therapeutically effective amount of thrombomodulin analogue to the subject in need of such treatment for one of the group of diseases consisting of haemophilia A, haemophilia B, haemophilia C, von Willebrandt disease (vWD), acquired von Willebrandt disease, Factor X deficiency, parahemophilia, hereditary disorders of the clotting factors I, II, V, or VII, haemorrhagic disorder due to circulating anticoagulants or acquired coagulation deficiency.

33. A method according to claim 32, wherein the subject is treated for one or more of the bleeding events selected from the group consisting of: intracranial or other CNS haemorrhage, bleeding in joints, microcapillaries, muscles, the gastrointestinal tract, the respiratory tract, the retroperitoneal space or soft tissues.

34. The method of claim 33, wherein the thrombomodulin analogue is administered to the subject at a time of the bleeding event.

35. The method according to claim 33, wherein the thrombomodulin analogue is administered in advance of an event where an increased risk of bleeding is expected.

36. The method of claim 35, wherein the event is surgery or a tooth extraction.

37. The method of claim 33, wherein the thrombomodulin analogue is administered to a subject that is refractory to blood/plasma transfusion or coagulation factor replacement therapy.

38. The method of claim 33, wherein the thrombomodulin analogue is administered in multiple doses selected from the group consisting of, once daily, bi-daily, every third, fourth, fifth, sixth or seven days over a total time period of less than one week to four weeks, and as a chronic administration.

39. The method of claim 33, wherein the thrombomodulin analogue is administered as parenteral application, as intravenous or subcutaneous application.

40. A method for screening for analogues of thrombomodulin suitable for the treatment of coagulopathy with hyperfibrinolysis, where the thrombomodulin exhibits one or more of the following features:
(i) a reduced binding affinity towards thrombin,
(ii) a reduced cofactor activity,
(iii) an increased TAFI activation activity, comprising the steps of:
a) making one or more amino acid substitution of the thrombomodulin sequence SEQ ID NO:1 or SEQ ID NO:3,
b) comparing the modified analogue with a control molecule with regard to one or more of the following characteristics:
ba) binding affinity to thrombin (KD value);
bb) cofactor activity;
bc) TAFI activation activity or TAFIa potential;
bd) ratio of TAFI activation activity and cofactor activity;
be) effect of protein oxidation;
bf) effect on clot lysis in time in an in vitro assay; or
bg) effect in a coagulation-associated animal model.

41. The method of claim 40, wherein the control molecules is a rabbit lung thrombomodulin or a soluble human thrombomodulin analogue.
